(19) United States
(12) Patent Application Publication
(10) Pub. No.: US 2010/0185183 A1
(43) Pub. Date: Jul. 22, 2010

(54) USER INTERFACE THAT DISPLAYS PENDING AND SELECTED PROGRAMMING FOR AN IMPLANTABLE MEDICAL DEVICE

(75) Inventors: Sarah B. Alme, Blaine, MN (US); Touby A. Drew, Golden Valley, MN (US); Ajinkya M. Joglekar, Maple Grove, MN (US); Jiaying Shen, Maple Grove, MN (US)

Correspondence Address: SHUMAKER & SIEFFERT , P.A, 1625 RADIO DRIVE , SUITE 300, WOODBURY, MN, 55125, US

(73) Assignee: Medtronic, Inc., Minneapolis, MN (US)

(21) Appl. No.: 12/691,592

(22) Filed: Jan. 21, 2010

Related U.S. Application Data
(60) Provisional application No. 61/146,594, filed on Jan. 22, 2009.

(51) Int. Cl. A61M 31/00; G06F 3/048

(52) U.S. Cl. 604/891.1; 715/772

(57) ABSTRACT

Techniques are described for simultaneously displaying representations, such as graphical representations, of both a selected dosing program of an implantable fluid delivery device and a pending dosing program of the implantable fluid delivery device. In one example, a system includes an implantable fluid delivery device that delivers fluid to a patient according to a selected dosing program, and a programmer device that includes a user interface comprising a display to present a graphical representation of doses of fluid to be delivered to a patient via an implantable fluid delivery device, and a processor that controls the user interface to simultaneously present on the display a first indication of a selected dosing program of the implantable fluid delivery device and a second indication of a pending dosing program of the implantable fluid delivery pump. A user may therefore compare the selected dosing program with the pending dosing program.

This application claims the benefit of U.S. Provisional Application No. 61/146,594, filed Jan. 22, 2009, which is hereby incorporated by reference in its entirety.

### TECHNICAL FIELD

This disclosure relates to programmers that control programming of implantable medical devices, such as implantable drug pumps.

### BACKGROUND

A variety of medical devices are used for chronic, e.g., long-term, delivery of therapy to patients suffering from a variety of conditions, such as chronic pain, tremor, Parkinson's disease, epilepsy, urinary or fecal incontinence, sexual dysfunction, obesity, spasticity, or gastroparesis. As examples, electrical stimulation generators are used for chronic delivery of electrical stimulation therapies such as cardiac pacing, neurostimulation, muscle stimulation, or the like. Pumps or other fluid delivery devices may be used for chronic delivery of therapeutic agents, such as drugs. Typically, such devices provide therapy continuously or periodically according to parameters contained within a program. A program may comprise respective values for each of a plurality of parameters, specified by a clinician. The devices may be implantable medical devices that receive the program from a programmer controlled by the clinician.

Examples of such implantable medical devices include implantable drug infusion pumps, implantable neurostimulators, implantable cardioverters, implantable cardiac pacemakers, implantable defibrillators, cochlear implants, and others that now exist or may exist in the future. These devices are intended to provide a patient with a therapeutic output to alleviate or assist with a variety of conditions. Typically such devices are implanted in a patient and provide a therapeutic output under specified conditions on a recurring basis.

One type of implantable fluid delivery device is a drug infusion device which can deliver a fluid medication to a patient at a selected site. A drug infusion device may be implanted at a location in the body of a patient and deliver a fluid medication through a catheter to a selected delivery site in the body. Drug infusion devices, such as implantable drug pumps, include fluid reservoirs that may be self-sealing and may be accessible through ports. A drug infusion device may be configured to deliver a therapeutic agent from the fluid reservoir to a patient according to a therapy program, which may, for example, specify a rate of delivery by the IMD of a fluid delivered to the patient.

Programmable implantable medical devices are typically programmed using an external programming device, sometimes known as a controller or programmer, which can communicate with the implanted medical device through well known techniques such as telemetry. An external controller, or programmer, can be used by a medical professional, for example, to change the therapeutic regimen by increasing or decreasing the amount of fluid medication delivered or by increasing or decreasing the intensity or timing or characteristic of an electrical stimulation signal. Typically, a medical professional interfaces with the external controller or programmer to set various parameters associated with the implantable medical device and then transmits, or downloads, those parameters to the implanted medical device. The external device may also record other information important to the delivery of the therapy. Some information may be stored in the implantable medical device and/or the external device. Such information may include, for example, patient information, implanted device information such as model, volume, implant location, length of catheter or lead, and other information specific to different devices.

### SUMMARY

In general, techniques are described for simultaneously displaying a selected dosing program and a pending dosing program of an implantable medical device (IMD), such as an implantable fluid delivery device. In general, this disclosure refers to a selected dosing program as a program according to which an IMD is currently operating to deliver fluid to a patient, a historical dosing program, an alternative dosing program, or other dosing program used as a basis for comparison for the pending dosing program.

The selected dosing program may be stored or encoded in a computer-readable medium of the IMD and/or a programming device. The selected dosing program may have been activated by a programming device in communication with the IMD. A “pending” dosing program, as referred to by this disclosure, is a dosing program according to which the IMD is not currently operating, e.g., delivering fluid. That is, a pending dosing program may either be a dosing program that has not yet been uploaded to the IMD by the programmer device, or a dosing program that is stored in the computer-readable medium of the IMD but not activated by the programmer device.

The implantable fluid delivery device delivers fluid at a rate for a duration of time according to a therapy schedule of the current dosing program. The therapy schedule of the current dosing program may include one or more “steps,” each of which may include a different rate and/or a different time duration. Over time, a clinician, doctor, patient, or other user of a programmer device may modify the current dosing program or design an entirely new dosing program.

The clinician may desire to observe the current dosing program of the implantable fluid delivery pump simultaneously with the pending dosing program, where the pending dosing program may comprise a modified version of the current dosing program. In accordance with the techniques described herein, the programmer device may provide a textual and/or graphical representation of the current dosing program simultaneously with the pending dosing program. The programmer device may further simultaneously display an additional representation, e.g., textual or graphical, of a third dosing program, or of multiple therapy schedules of a current or pending dosing program. In this manner, a user of the programmer device may observe changes to a therapy schedule of a dosing program and compare differences between two or more dosing programs or two or more therapy schedules at a particular point in time.

In one example, a method includes displaying, with a device for programming an implantable fluid delivery device, a first indication of a selected dosing program of the implantable fluid delivery device, and displaying, simultaneously with the first indication, a second indication of a pending dosing program of the implantable fluid delivery device.

In another example, a programmer device includes a user interface comprising a display to present a graphical representation of doses of fluid to be delivered to a patient via an implantable fluid delivery device, and a processor that controls the user interface to simultaneously present on the display a first indication of a selected dosing program of the implantable fluid delivery device and a second indication of a pending dosing program of the implantable fluid delivery pump.

In another example, a system includes an implantable fluid delivery pump that delivers fluid to a patient according to a selected dosing program, comprising a telemetry module, and a programmer device comprising: a user interface comprising a display to present a graphical representation of doses of fluid to be delivered to a patient via an implantable fluid delivery device, and a processor that controls the user interface to simultaneously present on the display a first indication of a selected dosing program of the implantable fluid delivery device and a second indication of a pending dosing program of the implantable fluid delivery pump.

In another example, a device includes means for displaying, with a device for programming an implantable fluid delivery device, a first indication of a selected dosing program of the implantable fluid delivery device, and means for displaying, simultaneously with the first indication, a second indication of a pending dosing program of the implantable fluid delivery device.

In another example, a computer-readable medium, such as a computer-readable storage medium, encodes instructions that cause a programmable processor to display a first indication of a selected dosing program of an implantable fluid delivery pump, and display, simultaneously with the first indication, a second indication of a pending dosing program of the implantable fluid delivery pump.

The details of one or more examples are set forth in the accompanying drawings and the description below. Other features, objects, and advantages will be apparent from the description and drawings, and from the claims.

### BRIEF DESCRIPTION OF DRAWINGS

FIG. 1 is a conceptual diagram illustrating an example of a fluid delivery system, which includes an implantable medical device that is configured to deliver a therapeutic agent to a patient via a catheter.

FIG. 2 is functional block diagram illustrating an example of an implantable fluid delivery device.

FIG. 3 is a functional block diagram illustrating example components of an external programmer for an implantable medical device.

FIG. 4 is a screenshot illustrating an example user interface screen presented by an external programmer when a user first begins interacting with the programmer.

FIGS. 5A and 5B are screenshots illustrating an example user interface screen presented by an external programmer for configuring an implantable medical device.

FIG. 6 is a screenshot illustrating an example user interface screen presented by an external programmer for editing patient information.

FIG. 7 is a screenshot illustrating an example user interface screen presented by an external programmer for editing fluid delivery pump and catheter information.

FIG. 8 is a screenshot illustrating an example user interface screen presented by an external programmer for editing drug information regarding a drug delivered by an implantable medical device.

FIG. 9 is a screenshot illustrating an example user interface screen for programming a therapy schedule of a dosing program.

FIG. 10 is a screenshot illustrating an example user interface screen that displays an infusion pattern based on user input received via the user interface of FIG. 9.

FIG. 11 is a screenshot illustrating an example user interface screen that allows a user to modify an infusion pattern.

FIG. 12 is a screenshot illustrating another example user interface screen by which a user may modify an infusion pattern.

FIG. 13 is a screenshot illustrating an example user interface screen for displaying current dosage information for a current dosing program simultaneously with pending dosage information for a pending dosing program.

FIG. 14 is a block diagram illustrating an example graph that graphically represents various dosing programs with distinct shading styles.

FIG. 15 is a block diagram illustrating an example graph that graphically represents various dosing programs with distinct line styles.

FIG. 16 is a block diagram illustrating an example graph that illustrates three graphical representations of three distinct dosing programs.

FIG. 17A is a screenshot of an example user interface that displays current settings of an implantable medical device simultaneously with pending settings of the implantable medical device.

FIG. 17B is a screenshot of another example user interface that displays current settings of IMD 12 simultaneously with pending settings of IMD 12.

FIG. 18 is a screenshot illustrating additional settings of the implantable medical device of FIG. 17A that are displayed after the user has scrolled to a different set of information with scroll buttons of the user interface.

FIG. 19 is a flowchart illustrating an example method for displaying a first indication of a pending dosing program and a second indication of a current dosing program simultaneously.

FIG. 20 is a flowchart illustrating an example method for displaying an indication of a first dosing program and an indication of a second dosing program simultaneously.

FIG. 21 is a screenshot illustrating an example user interface for displaying three dosing programs simultaneously.

### DETAILED DESCRIPTION

Medical devices are useful for treating, managing or otherwise controlling various patient conditions or disorders, such as, but not limited to, pain (e.g., chronic pain, post-operative pain or peripheral and localized pain), tremor, movement disorders (e.g., Parkinson's disease), diabetes, epilepsy, neuralgia, chronic migraines, urinary or fecal incontinence, sexual dysfunction, obesity, gastroparesis, mood disorders, or other disorders. Some medical devices may be configured to deliver one or more therapeutic agents, alone or in combination with other therapies, such as electrical stimulation, to one or more target sites within a patient. For example, in some cases, a medical device may deliver one or more pain-relieving drugs to patients with chronic pain, insulin to a patient with diabetes, or other fluids to patients with different disorders. The medical device may be implanted in the patient for chronic therapy delivery (e.g., longer than a temporary, trial basis) or temporary delivery.

As used in this disclosure, the term dosing program generally refers to a program sent to an implantable fluid delivery device by a device for programming the implantable fluid delivery device to cause the implantable fluid delivery device to deliver fluid at a certain rate at a certain time. The dosing program may include, for example, definitions of a priming bolus, a bridging phase, a supplemental bolus, and a therapy schedule. A dosing program may include additional information, such as patient information, permissions for a user to add a supplemental bolus, historical therapy schedules, fluid or drug information, or other information. The term therapy schedule may generally refer to a rate (which may be zero) at which to administer fluid, or a drug or drug combination within the fluid, at a specific time to a patient. In particular, the therapy schedule defines one or more programmed doses, which may be periodic or aperiodic, each dose including a rate of fluid delivery and a time duration for which to deliver the dose. Dose generally refers to the amount of drug delivered over a period of time, and may change over the course of a therapy schedule such that a drug may be delivered at different rates at different times.

A priming bolus refers to a bolus delivered by the implantable fluid delivery device to move the fluid to the distal tip of the catheter, i.e., the tip of the catheter that is remote from the reservoir and internal tubing, if applicable. Once the fluid is primed to the distal tip of the catheter, the IMD is ready to deliver fluid to the patient from the distal tip, e.g., via one or more fluid outlets at or near the distal tip. The device delivers the priming bolus during a priming phase to prepare the device for delivery of the fluid to the patient. A priming phase may generally refer to a delivery phase during which fluid is moved toward the distal tip of the catheter. Depending on the structure of the IMD, the priming phase may involve delivery of fluid from the reservoir, through internal tubing coupled to the reservoir, and to the tip of a catheter coupled to the tubing, such that the fluid is in a position to be infused into the patient when desired.

An implantable fluid delivery device also may perform a bridge when a new fluid is inserted into a reservoir of the device while an old fluid is still present in the device, e.g., within internal tubing of the device and/or within a catheter connected to the device. The bridge is performed to define a rate at which to deliver the old fluid until the old fluid is completely delivered out of the catheter and to the patient such that the device contains only the new fluid. A supplemental bolus is a bolus administered to the patient outside of the therapy schedule. The terms independent bolus, one-time bolus, and therapeutic bolus may also be used relatively interchangeably in this disclosure to refer to a supplemental bolus. In one example, the implantable fluid delivery device may administer a supplemental bolus before the implantable fluid delivery device begins administering doses of fluid according to the therapy schedule. In another example, the implantable fluid delivery device may administer a supplemental bolus during the therapy schedule, e.g., to override or supplement the therapy schedule in response to clinician instruction or patient request.

The implantable fluid delivery device delivers fluid at a rate for a duration of time according to a therapy schedule of the current dosing program. The therapy schedule of the current dosing program may include one or more “steps,” each of which may include a different rate and/or a different time duration. Over time, a clinician, doctor, patient, or other user of a programmer device may modify the dosing program. For example, a clinician may determine that a patient requires a higher dose of a drug for a given period of time, such as a 24-hour period. The clinician may also determine that the patient is more active at certain times of the day than others, such that the patient requires a higher dose during those times than at other times of the day. The clinician may also determine that the patient has been experiencing excessive dose symptoms, so the clinician may reduce the dose delivered to the patient.

The clinician may desire to observe the current dosing program or another selected dosing program of the implantable fluid delivery pump simultaneously with the pending dosing program. In other examples, the clinician may request to view any selected dosing program concurrently with the pending dosing program. Although the programmer device may generally display any selected dosing program, e.g., a current dosing program, an alternative pending dosing program, a historical dosing program, or other dosing program concurrently with the pending dosing program, this disclosure generally refers to a current dosing program by way of example. The pending program may be a program that has not yet been selected to control the fluid delivery pump. In some cases, the pending program has been defined using a patient programmer but is under consideration and has not yet been downloaded to the fluid delivery pump. In other cases, the pending program may have been downloaded to the fluid delivery pump, but has not yet been selected or otherwise activated to control the fluid delivery pump.

In accordance with the techniques described herein, the programmer device may provide a textual and/or graphical representation of the current dosing program (or another selected dosing program) simultaneously with the pending dosing program. In this manner, the clinician may immediately compare dosage rates, quantities, and durations between the current dosing program and the pending dosing program. The clinician may therefore modify the pending dosing program if necessary, e.g., if the clinician observes that too much or too little new drug will be administered to the patient too quickly or slowly each day. The programmer device may therefore assist the clinician in avoiding overdose or underdose of the patient.

The programmer device may further display a plurality of dosing programs. For example, the programmer device may simultaneously display a historical set of dosing programs. The historical dosing programs may comprise one or more dosing programs that were previously applied by the IMD but are no longer active. One or more of the historical dosing programs may remain encoded in a computer-readable medium of the IMD, or may have been erased from the computer-readable medium of the IMD. The clinician and the patient may observe the historical set to determine which dosing programs were most effective, in order to program the implantable fluid delivery pump to be most effective for the patient. The historical set may also further assist the clinician in avoiding overdose or underdose by observing a larger sample and longer history of dosages of a drug for the patient.

As another example, the programmer device may simultaneously display multiple, i.e., two or more, dosing programs, or multiple therapy schedules of a single dosing program. This disclosure refers to a “therapy schedule” as a series of programmed doses of fluid, each of which are delivered by the IMD at a particular rate for a particular time. As one example, the clinician may determine that during weekdays, the implantable fluid delivery pump should operate according to a first therapy schedule, but during weekends, the implantable fluid delivery pump should operate according to a second therapy schedule. The programmer device may allow the clinician both to program the implantable fluid delivery pump according to a dosing program that comprises two or more different therapy schedules, and the programmer device may simultaneously display each of the therapy schedules, e.g., with a user interface. In this manner, the programmer device permits a clinician to observe differences in rate of fluid delivery by the IMD over time. The programmer device may also permit the clinician to modify or refine therapy schedules in comparison to other therapy schedules, or dosing programs in comparison to other dosing programs. In this manner, the programmer device may display a first indication of a current dosing program of an implantable fluid delivery pump and, simultaneously, display a second indication of a pending dosing program of the implantable fluid delivery pump.

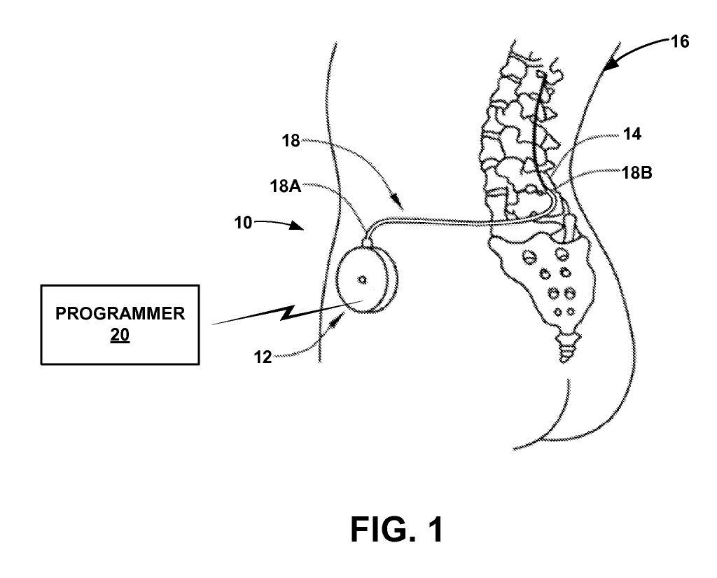

FIG. 1 is a conceptual diagram illustrating an example of a therapy system 10, which includes IMD 12 configured to deliver at least one therapeutic agent, such as a pharmaceutical agent, insulin, pain relieving agent, anti-inflammatory agent, gene therapy agent, or the like, to a target site within patient 16 via catheter 18, which is coupled to IMD 12. In one example, catheter 18 may comprise a plurality of catheter segments. In other examples, catheter 18 may be a unitary catheter. In the example of FIG. 1, the therapeutic agent is a therapeutic fluid. IMD 12 may be, for example, an implantable fluid delivery device that delivers therapeutic agents in fluid form to patient 16. In the example shown in FIG. 1, the target site is proximate to spinal cord 14 of patient 16. A proximal end 18A of catheter 18 is coupled to IMD 12, while a distal end 18B of catheter 18 is located proximate to the target site. Therapy system 10 also includes external programmer 20, which wirelessly communicates with IMD 12 as needed, such as to provide or retrieve therapy information or control aspects of therapy delivery (e.g., modify the therapy parameters, turn IMD 12 on or off, and so forth). While patient 16 is generally referred to as a human patient, other mammalian or non-mammalian patients are also contemplated.

Generally, IMD 12 has an outer housing that is constructed of a biocompatible material that resists corrosion and degradation from bodily fluids, such as titanium or biologically inert polymers. IMD 12 may be implanted within a subcutaneous pocket close to the therapy delivery site. For example, in the example shown in FIG. 1, IMD 12 is implanted within an abdomen of patient 16. In other examples, IMD 12 may be implanted within other suitable sites within patient 16, which may depend, for example, on the target site within patient 16 for the delivery of the therapeutic agent.

Catheter 18 may be coupled to IMD 12 either directly or with the aid of an extension (not shown in FIG. 1). In the example shown in FIG. 1, catheter 18 traverses from the implant site of IMD 12 to one or more target sites proximate to spine 14. Catheter 18 is positioned such that one or more fluid delivery outlets of catheter 18 are proximate to the one or more target sites within patient 16. IMD 12 delivers a therapeutic agent to the one or more target sites proximate to spinal cord 14 with the aid of catheter 18. For example, IMD 12 may be configured for intrathecal drug delivery into the intrathecal space or epidural space surrounding spinal cord 14. The intrathecal space is within the subarachnoid space of spinal cord 14, which is past the epidural space and dura mater and through the theca of spinal cord 14.

Therapy system 10 may be used, for example, to reduce pain experienced by patient 16. IMD 12 may deliver one or more therapeutic agents to patient 16 according to one or more dosing programs that set forth different therapy parameters, such as a therapy schedule specifying programmed doses, dose rates for the programmed dose, and specific times to deliver the programmed doses. In some examples, the therapeutic agent may be a liquid. The dosing programs may be may be a part of a program group for therapy, where the group includes a plurality of therapy schedules. In some examples, IMD 12 may be configured to deliver a therapeutic agent to patient 16 according to different therapy schedules on a selective basis. IMD 12 may include a memory to store one or more therapy programs, instructions defining the extent to which patient 16 may adjust therapy parameters, switch between dosing programs, or undertake other therapy adjustments. Patient 16 may select and/or generate additional dosing programs for use by IMD 12 via external programmer 20 at any time during therapy or as designated by the clinician.

In some examples, multiple catheters 18 may be coupled to IMD 12 to target the same or different tissue sites within patient 16. Thus, although a single catheter 18 is shown in FIG. 1, in other examples, system 10 may include multiple catheters or catheter 18 may define multiple lumens for delivering different therapeutic agents to patient 16 or for delivering a therapeutic agent to different tissue sites within patient 16. Accordingly, in some examples, IMD 12 may include a plurality of reservoirs for storing more than one type of therapeutic agent. In some examples, IMD 12 may include a single long tube that contains the therapeutic agent in place of a reservoir. However, for ease of description, an IMD 12 including a single reservoir is primarily discussed herein.

Programmer 20 is an external computing device that is configured to wirelessly communicate with IMD 12. For example, programmer 20 may be a clinician programmer that the clinician uses to communicate with IMD 12. Alternatively, programmer 20 may be a patient programmer that allows patient 16 to view and modify therapy parameters. The clinician programmer may include additional or alternative programming features than the patient programmer. For example, more complex or sensitive tasks may only be allowed by the clinician programmer to prevent patient 16 from making undesired changes to the operation of IMD 12.

Programmer 20 may be a hand-held computing device that includes a display viewable by the user and a user input mechanism that can be used to provide input to programmer 20. For example, programmer 20 may include a display screen (e.g., a liquid crystal display or a light emitting diode display) that presents information to the user. In addition, programmer 20 may include a keypad, buttons, a peripheral pointing device, touch screen, voice recognition, or another input mechanism that allows the user to navigate though the user interface of programmer 20 and provide input.

If programmer 20 includes buttons and a keypad, the buttons may be dedicated to performing a certain function, i.e., a power button, or the buttons and the keypad may be soft keys that change in function depending upon the section of the user interface currently viewed by the user. Alternatively, the screen (not shown) of programmer 20 may be a touch screen that allows the user to provide input directly to the user interface shown on the display. The user may use a stylus or their finger to provide input to the display.

In other examples, rather than being a handheld computing device or a dedicated computing device, programmer 20 may be a larger workstation or a separate application within another multi-function device. For example, the multi-function device may be a cellular phone, personal computer, laptop, workstation computer, or personal digital assistant that can be configured to an application to simulate programmer 20. Alternatively, a notebook computer, tablet computer, or other personal computer may enter an application to become programmer 20 with a wireless adapter connected to the personal computer for communicating with IMD 12.

When programmer 20 is configured for use by the clinician, programmer 20 may be used to transmit initial programming information to IMD 12. This initial information may include system 10 hardware information such as the type of catheter 18, the position of catheter 18 within patient 16, the type of therapeutic agent(s) delivered by IMD 12, a baseline orientation of at least a portion of IMD 12 relative to a reference point, therapy parameters of therapy programs stored within IMD 12 or within programmer 20, and any other information the clinician desires to program into IMD 12.

The clinician uses programmer 20 to program IMD 12 with one or more therapy programs that define the therapy delivered by IMD 12. During a programming session, the clinician may determine one or more dosing programs that may provide effective therapy to patient 16. Patient 16 may provide feedback to the clinician as to the efficacy of a specific program being evaluated or desired modifications to the dosing program. Once the clinician has identified one or more programs that may be beneficial to patient 16, patient 16 may continue the evaluation process and determine which dosing program or therapy schedule best alleviates the condition of patient 16 or otherwise provides efficacious therapy to patient 16. The dosing program information may set forth therapy parameters, such as different predetermined dosages of the therapeutic agent (e.g., a dose amount), the rate of delivery of the therapeutic agent (e.g., rate of delivery of the fluid), the maximum acceptable dose, a time interval between successive patient-initiated boluses (e.g., a lock-out interval), a maximum dose that may be delivered over a given time interval, and so forth. IMD 12 may include a feature that prevents dosing the therapeutic agent in a manner inconsistent with the therapy program. Programmer 20 may assist the clinician in the creation/identification of therapy programs by providing a methodical system of identifying potentially beneficial therapy parameters.

A dosage of a therapeutic agent, such as a drug, may be expressed as an amount of drug, e.g., measured in milligrams, provided to the patient over a particular time interval, e.g., per day or twenty-four hour period. This dosage amount may convey to the caregiver an indication of the probable efficacy of the drug and the possibility of side effects of the drug. In general, a sufficient amount of the drug should be administered in order to have a desired therapeutic effect, such as pain relief. However, the amount of the drug administered to the patient should be limited to a maximum amount, such as a maximum daily dose, in order not to avoid potential side effects. Program information specified by a user via programmer 20 may be used to control dosage amount, dosage rate, maximum dose for a given time interval (e.g., daily), or other parameters associated with delivery of a drug or other fluid by IMD 12.

In some cases, programmer 20 may also be configured for use by patient 16. When configured as the patient programmer, programmer 20 may have limited functionality in order to prevent patient 16 from altering critical functions or applications that may be detrimental to patient 16. In this manner, programmer 20 may only allow patient 16 to adjust certain therapy parameters or set an available range for a particular therapy parameter. In some cases, a patient programmer may permit the patient to control IMD 12 to deliver a supplemental, patient bolus, if permitted by the applicable therapy program administered by the IMD, e.g., if delivery of a patient bolus would not violate a lockout interval or maximum dosage limit. Programmer 20 may also provide an indication to patient 16 when therapy is being delivered or when IMD 12 needs to be refilled or when the power source within programmer 20 or IMD 12 need to be replaced or recharged.

Whether programmer 20 is configured for clinician or patient use, programmer 20 may communicate to IMD 12 or any other computing device via wireless communication. Programmer 20, for example, may communicate via wireless communication with IMD 12 using radio frequency (RF) telemetry techniques known in the art. Programmer 20 may also communicate with another programmer or computing device via a wired or wireless connection using any of a variety of local wireless communication techniques, such as RF communication according to the 802.11 or Bluetooth specification sets, infrared (IR) communication according to the IRDA specification set, or other standard or proprietary telemetry protocols. Programmer 20 may also communicate with another programming or computing device via exchange of removable media, such as magnetic or optical disks, or memory cards or sticks. Further, programmer 20 may communicate with IMD 12 and another programmer via remote telemetry techniques known in the art, communicating via a local area network (LAN), wide area network (WAN), public switched telephone network (PSTN), or cellular telephone network, for example.

In other applications of therapy system 10, the target therapy delivery site within patient 16 may be a location proximate to sacral nerves (e.g., the S2, S3, or S4 sacral nerves) in patient 16 or any other suitable nerve, organ, muscle or muscle group in patient 16, which may be selected based on, for example, a patient condition. For example, therapy system 10 may be used to deliver a therapeutic agent to tissue proximate to a pudendal nerve, a perineal nerve or other areas of the nervous system, in which cases, catheter 18 would be implanted and substantially fixed proximate to the respective nerve. As further examples, catheter 18 may be positioned to deliver a therapeutic agent to help manage peripheral neuropathy or post-operative pain mitigation, ilioinguinal nerve stimulation, intercostal nerve stimulation, gastric stimulation for the treatment of gastric mobility disorders and obesity, muscle stimulation, for mitigation of other peripheral and localized pain (e.g., leg pain or back pain). As another example, catheter 18 may be positioned to deliver a therapeutic agent to a deep brain site or within the heart (e.g., intraventricular delivery of the agent). Delivery of a therapeutic agent with the brain may help manage any number of disorders or diseases. Example disorders may include depression or other mood disorders, dementia, obsessive-compulsive disorder, migraines, obesity, and movement disorders, such as Parkinson's disease, spasticity, and epilepsy. Catheter 18 may also be positioned to deliver insulin to a patient with diabetes.

Examples of therapeutic agents IMD 12 may be configured to deliver include, but are not limited to, insulin, morphine, other pain mitigating pharmaceutical agents, genetic agents, antibiotics, nutritional fluids, analgesics, hormones or hormonal drugs, gene therapy drugs, anticoagulants, cardiovascular medications or chemotherapeutics.

In accordance with the techniques described herein, programmer 20 may display simultaneous representations of two or more dosing programs for IMD 12. That is, programmer 20 may display a first representation of a first set of dosage information for a current dosing program of IMD 12, which dosing program IMD 12 is currently administering to patient 16. Programmer 20 may simultaneously display a second representation of a second set of dosage information for a pending dosing program, according to which programmer 20 may program IMD 12. A user of programmer 20 may therefore observe the current program simultaneously with the pending program to view differences in quantity of drug or fluid being administered, times at which the drug will be administered, durations of various dosage rates, total quantities being administered over a period, e.g., 24 hours, or other information. Such information may assist a clinician or other user of programmer 20 to recognize and prevent overdose or underdose situations for patient 16.

In one example, programmer 20 may simultaneously display a plurality of representations corresponding to a plurality of programs. For example, programmer 20 may display a history of dosing programs. A clinician and a patient may review the history to determine which dosing programs were most effective. The clinician and the patient may correlate the graphical information with patient activity information to determine whether the dosing program should be changed for certain days, e.g., increased dosage during weekdays and decreased dosage during weekends. As another example, programmer 20 may display multiple dosing programs as a pending dosing program and an alternative dosing program. The pending dosing program may correspond to a dosing program to be administered by IMD 12 during weekdays and the alternative dosing program may correspond to a dosing program to be administered by IMD 12 during weekends. In this manner, the user may observe differences in duration of dosing periods, quantities of drug/fluid administered at different times, differences in total quantities of drug administered for a common period of time, or other observable features. The user may use this information to assist in modifying dosing programs, reviewing performance of dosing programs, or for other purposes.

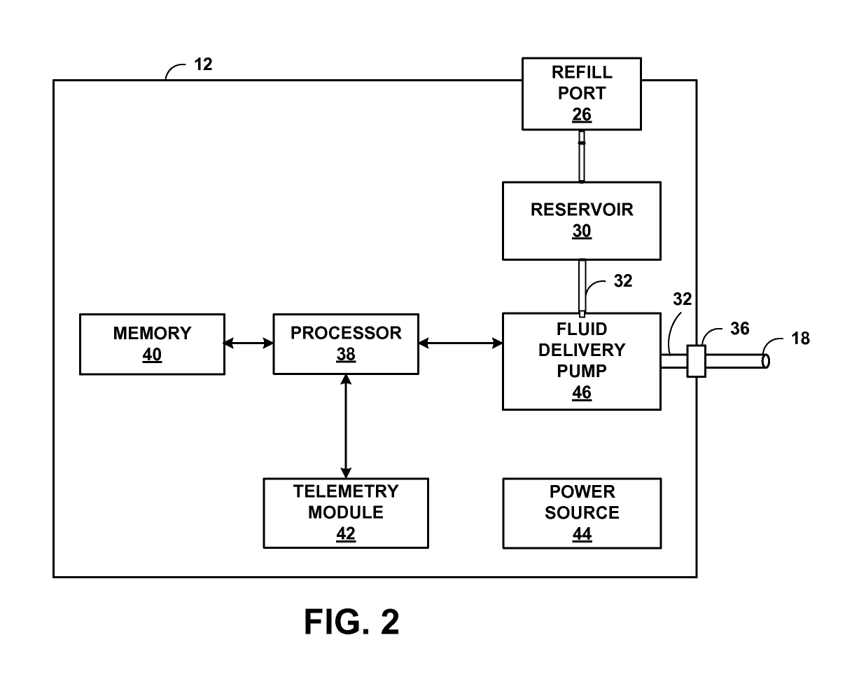

FIG. 2 is a functional block diagram illustrating components of an example of IMD 12, which includes refill port 26, reservoir 30, processor 38, memory 40, telemetry module 42, power source 44, fluid delivery pump 46, internal tubing 32, and catheter access port 36. Fluid delivery pump 46 may be a mechanism that delivers a therapeutic agent in some metered or other desired flow dosage to the therapy site within patient 16 from reservoir 30 via the catheter 18. Refill port 26 may comprise a self-sealing membrane to prevent loss of therapeutic agent delivered to reservoir 30 via refill port 26. After a delivery system, e.g., a hypodermic needle, penetrates the membrane of refill port 26, the membrane may seal shut when the needle is removed from refill port 26.

Internal tubing 32 is a segment of tubing that runs from reservoir 30, around or through fluid delivery pump 46 to catheter access port 36. In one example, fluid delivery pump 46 may be a squeeze pump that squeezes internal tubing 32 in a controlled manner, e.g., such as a peristaltic pump, to progressively move fluid from reservoir 30 to the distal end of catheter 18 and then into the patient according to parameters specified by a set of program information. Fluid delivery pump 46 may, in other examples, comprise a an axial pump, a centrifugal pump, a pusher plate, a piston-driven pump, or other means for moving fluid through internal tubing 32 and catheter 18.

Processor 38 controls the operation of fluid delivery pump 46 with the aid of instructions associated with program information that is stored in memory 40. For example, the instructions may define dosing programs that specify the amount of a therapeutic agent that is delivered to a target tissue site within patient 16 from reservoir 30 via catheter 18. The instructions may further specify the time at which the agent will be delivered and the time interval over which the agent will be delivered. The amount of the agent and the time over which the agent will be delivered are a function of the dosage rate at which the fluid is delivered. The therapy programs may also include other therapy parameters, such as the frequency of bolus delivery, the type of therapeutic agent delivered if IMD 12 is configured to deliver more than one type of therapeutic agent), and so forth. Components described as processors within IMD 12, external programmer 20, or any other device described in this disclosure may each comprise one or more processors, such as one or more microprocessors, digital signal processors (DSPs), application specific integrated circuits (ASICs), field programmable gate arrays (FPGAs), programmable logic circuitry, or the like, either alone or in any suitable combination.

Memory 40 may include any volatile or non-volatile media, such as a random access memory (RAM), read only memory (ROM), non-volatile RAM (NVRAM), electrically erasable programmable ROM (EEPROM), flash memory, and the like. As mentioned above, memory 40 may store program information including instructions for execution by processor 38, such as, but not limited to, therapy programs, historical therapy programs, timing programs for delivery of fluid from reservoir 30 to catheter 18, and any other information regarding therapy of patient 16. A program may indicate the bolus size or flow rate of the drug, and processor 38 may accordingly deliver therapy. Memory 40 may include separate memories for storing instructions, patient information, therapy parameters (e.g., grouped into sets referred to as dosing programs), therapy adjustment information, program histories, and other categories of information such as any other data that may benefit from separate physical memory modules. Therapy adjustment information may include information relating to timing, frequency, rates and amounts of patient boluses or other permitted patient modifications to therapy. In some examples, memory 40 stores program instructions that, when executed by processor 38, cause IMD 12 and processor 38 to perform the functions attributed to them in this disclosure.

Telemetry module 42 in IMD 12, as well as telemetry modules in other devices described herein, such as programmer 20, may accomplish communication by RF communication techniques. In addition, telemetry module 42 may communicate with programmer 20 via proximal inductive interaction of IMD 12 with external programmer 20. Accordingly, telemetry module 42 may send information to external programmer 20 on a continuous basis, at periodic intervals, or upon request from the programmer. Processor 38 controls telemetry module 42 to send and receive information. Wireless telemetry may be accomplished by RF communication or proximal inductive interaction of IMD 12 with external programmer 20.

Power source 44 delivers operating power to various components of IMD 12. Power source 44 may include a small rechargeable or non-rechargeable battery and a power generation circuit to produce the operating power. In the case of a rechargeable battery, recharging may be accomplished through proximal inductive interaction between an external charger and an inductive charging coil within IMD 12. In some examples, power requirements may be small enough to allow IMD 12 to utilize patient motion and implement a kinetic energy-scavenging device to trickle charge a rechargeable battery. In other examples, traditional batteries may be used for a limited period of time. As a further alternative, an external inductive power supply could transcutaneously power IMD 12 whenever measurements are needed or desired.

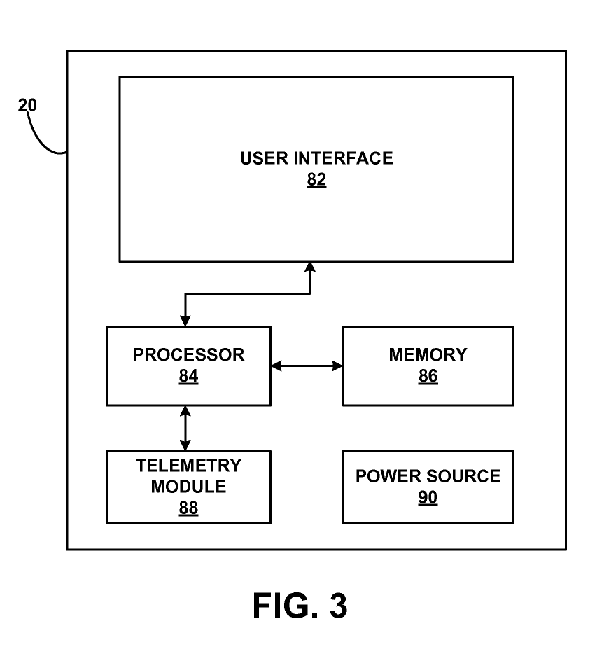

FIG. 3 is a functional block diagram illustrating various components of an external programmer 20 for IMD 12. As shown in FIG. 3, external programmer 20 includes processor 84, memory 86, telemetry module 88, user interface 82, and power source 90. A clinician or patient 16 interacts with user interface 82 in order to manually change the parameters of a dosing program, change dosing programs within a group of programs, view therapy information, view historical therapy regimens, establish new therapy regimens, or otherwise communicate with IMD 12 or view programming information.

User interface 82 may include a screen and one or more input buttons, as discussed in greater detail below, that allow external programmer 20 to receive input from a user. Alternatively, user interface 82 may additionally or only utilize a touch screen display, as in the example of clinician programmer 60. The screen may be a liquid crystal display (LCD), dot matrix display, organic light-emitting diode (OLED) display, touch screen, or any other device capable of delivering and/or accepting information. For visible posture state indications, a display screen may suffice. For audible and/or tactile posture state indications, programmer 20 may further include one or more audio speakers, voice synthesizer chips, piezoelectric buzzers, or the like.

Input buttons for user interface 82 may include a touch pad, increase and decrease buttons, emergency shut off button, and other buttons needed to control the therapy, as described above with regard to patient programmer 20. Processor 84 controls user interface 82, retrieves data from memory 86 and stores data within memory 86. Processor 84 also controls the transmission of data through telemetry module 88 to IMD 12. Memory 86 includes operation instructions for processor 84 and data related to patient 16 therapy.

User interface 82 may be configured to present therapy program information to the user. User interface 82 enables a user to program IMD 12 in accordance with one or more dosing programs, therapy schedules, or the like. As discussed in greater detail below, a user such as a clinician, physician or other caregiver may input patient information, drug information, therapy schedules, priming information, bridging information, drug/IMD implant location information, or other information to programmer 20 via user interface 82. In addition, user interface 82 may display therapy program information as graphical bar graphs or charts, numerical spread sheets, or in any other manner in which information may be displayed. Further, user interface 82 may present nominal or suggested therapy parameters that the user may accept via user interface 82.

User interface 82 may be configured to display a location of IMD 12 within patient 16, as well as a location of fluid within IMD 12. User interface 82 may display a representation of the location of the fluid overlaid with a representation of IMD 12. The representation may be a graphical representation. In some cases, the graphical representation may be a pictorial representation. As described in greater detail herein, user interface 82 may display a representation of progress of a delivery phase of IMD 12. User interface 82 may simultaneously display a representation of fluid corresponding to the represented progress of the delivery phase at a location within IMD 12. For example, user interface 82 may indicate the location of the fluid within IMD 12 at the time corresponding to the progress of the delivery phase. User interface 82 may present a graphical representation of a portion of IMD 12, and present an indication of a location of fluid within the graphical representation of IMD 12 corresponding to the delivery phase of IMD 12.

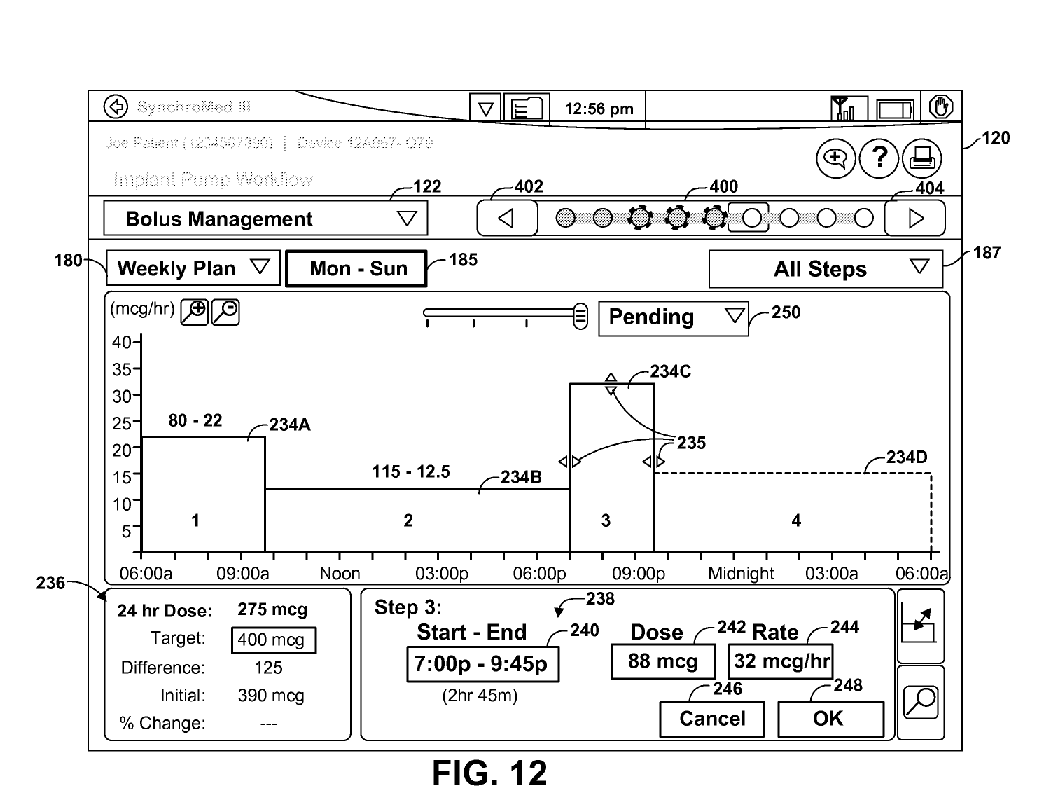

In some examples, the location may be indicated, in part, by the position of the distal-most portion of the fluid column defined by the fluid extending toward the distal end of the catheter 18 within IMD 12. User interface 82 may display a representation of the delivery phase as a simulation or during the actual delivery phase of IMD 12. As one example, and as further discussed with respect to, e.g., FIGS. 12A-12C, user interface 82 may display a representation of a reservoir, internal tubing, and a catheter of IMD 12, and display the location of the fluid during the priming phase as being in the reservoir, the internal tubing, and/or the catheter of IMD 12. The therapy program information may also be stored within memory 86 periodically during therapy, whenever external programmer 20 communicates within IMD 12, or only when the user desires to use or update the therapy program information.

Telemetry module 88 allows the transfer of data to and from IMD 12. Telemetry module 88 may communicate automatically with IMD 12 at a scheduled time or when the telemetry module detects the proximity of IMD 12. Alternatively, telemetry module 88 may communicate with IMD 12 when signaled by a user through user interface 82. To support RF communication, telemetry module 88 may include appropriate electronic components, such as amplifiers, filters, mixers, encoders, decoders, and the like. Power source 90 may be a rechargeable battery, such as a lithium ion or nickel metal hydride battery. Other rechargeable or conventional batteries may also be used. In some cases, external programmer 20 may be used when coupled to an alternating current (AC) outlet, i.e., AC line power, either directly or via an AC/DC adapter.

In some examples, external programmer 20 may be configured to recharge IMD 12 in addition to programming IMD 12. Alternatively, a recharging device may be capable of communication with IMD 12. Then, the recharging device may be able to transfer programming information, data, or any other information described herein to IMD 12. In this manner, the recharging device may be able to act as an intermediary communication device between external programmer 20 and IMD 12. The techniques described herein may be communicated between IMD 12 via any type of external device capable of communication with IMD 12.

External programmer 20 is an example of a programmer device that includes a user interface comprising a display to present a graphical representation of doses of fluid to be delivered to a patient via an implantable fluid delivery device and a processor that controls the user interface to simultaneously present on the display a first indication of a selected dosing program of the implantable fluid delivery device and a second indication of a pending dosing program of the implantable fluid delivery pump.

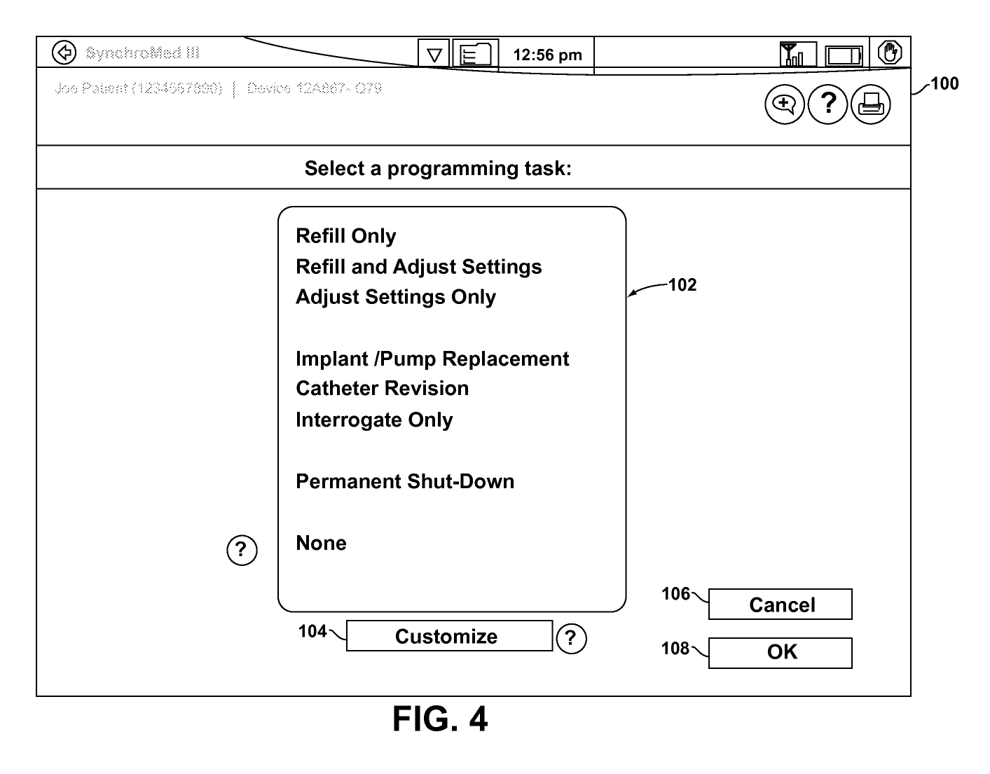

FIG. 4 is a screenshot illustrating an example user interface screen 100 presented by programmer 20 when a user first begins interacting with programmer 20. User interface screen 100 may be displayed by user interface 82 of programmer 20. When a user begins a session of working with programmer 20, programmer 20 presents user interface screen 100 to the user. The user may select from a variety of tasks 102 to perform using programmer 20. Tasks 102 of FIG. 4 depict example tasks “refill only,” “refill and adjust settings,” “adjust settings only,” “implant/pump replacement,” “catheter revision,” “interrogate only,” “permanent shut-down,” and “none.” Other examples may include additional or fewer tasks for the user to perform.

In one example, processor 84 determines, from the selection of tasks 102, a series of task screens to present to the user of programmer 20 via user interface 82. Processor 84 may cause user interface 82 to present one or more task screens, each of which may receive one or more pieces of data from the user, in order to program IMD 12 according to the selected one of tasks 102. Programmer 20 may implement more task screens than are necessary for any one of tasks 102, therefore programmer 20 may not display unnecessary screens to the user. In this manner, programmer 20 may assist a user in efficiently programming IMD 12 by requesting relevant data without requesting unnecessary data. Programmer 20 may therefore minimize the number of screens that are displayed based on a selection from tasks 102.

The user may select one of tasks 102 from user interface screen 100. For example, the user may select one of the tasks using a pointer controlled by a mouse. As another example, the user may select one of the tasks by touching the task on a touch-screen interface, e.g., with a finger or a stylus. The user may select Cancel button 106 to end the current session or, after selecting one of tasks 102, the user may select OK button 108 to perform the task. Upon receiving a selection of OK button 108, the user interface presents a different display screen to enable the user to perform the task with programmer 20. The user may also select Customize button 104 to customize the task selected from list 102, e.g., one or more parts or sub-parts of the task selected from list 102. In this manner, the user may select which of the one or more task screens are displayed.

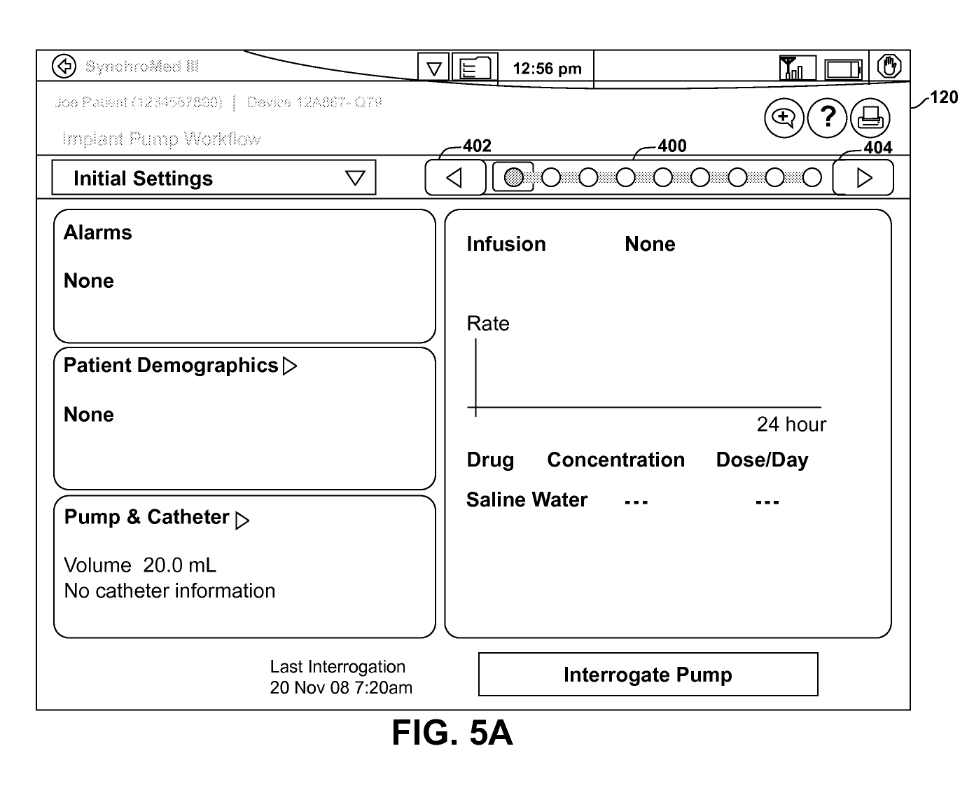

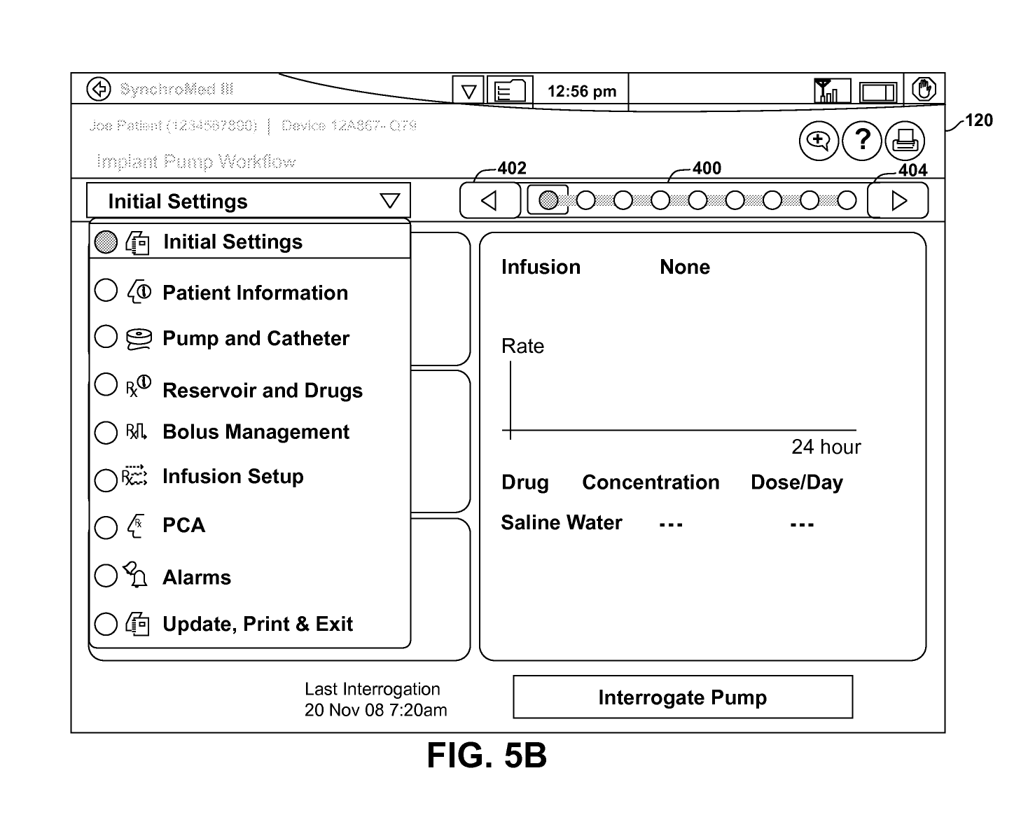

FIGS. 5A and 5B are screenshots illustrating an example user interface screen 120 for configuring IMD 12. User interface screen 120 may be displayed by user interface 82 of programmer 20. User interface screen 120 may be displayed, e.g., when a user selects the “implant/pump replacement” task from tasks 102 and then selects OK button 108 of user interface screen 100 (FIG. 4). FIG. 5A depicts current information for the current settings of an IMD, such as IMD 12, associated with the program. For example, the information includes alarms, patient demographics, pump and catheter information, drug information, and an infusion pattern in the example of FIG. 5A. The user may select Interrogate Pump button 124 to populate programmer 20 with data stored on IMD 12. The user may elect to do this when IMD 12 has current patient data, but programmer 20 does not. For example, IMD 12 may have been programmed with a different programmer than programmer 20. The user may also select Interrogate Pump button 124 to retrieve data from IMD 12 that IMD 12 has collected during operation within patient 16.

The user may edit settings of the program by selecting various options from drop-down menu 122. FIG. 5B illustrates the options available in the example, which includes “initial settings,” “patient information,” “pump and catheter,” “reservoir and drugs,” “bolus management,” “infusion setup,” “PCA,” “alarms,” and “update, print, and exit.” When a user selects any of these options from drop-down menu 122, programmer 20 updates and refreshes user interface screen 120 to illustrate settings and options for the selected option, as discussed in greater detail below.

FIGS. 5A and 5B also include programming status indicator 400, left navigation arrow 402, and right navigation arrow 404. Programming status indicator 400 informs the user of where the user is in the programming process, e.g., in the process of developing a pending dosing program for IMD 12. Processor 84 may, according to instructions encoded within memory 86 of programmer 20, determine which screens are necessary to program IMD 12 based on input received with the example user interface of FIG. 4. The user may also navigate between screens using left navigation arrow 402 and right navigation arrow 404. In addition, the user may jump to a particular screen by selecting the screen from drop-down menu 122. When the user selects left navigation arrow 402, processor 84 may cause user interface screen 120 to display a previous screen in the process of programming IMD 12. Similarly, when the user selects right navigation arrow 404, processor 84 may cause user interface screen 120 to display a next screen in the process of programming IMD 12. Status indicator 400 may indicate whether the user has entered sufficient information in each screen, which screen the user is currently viewing, how many screens are remaining, or other information regarding the programming status of IMD 12.

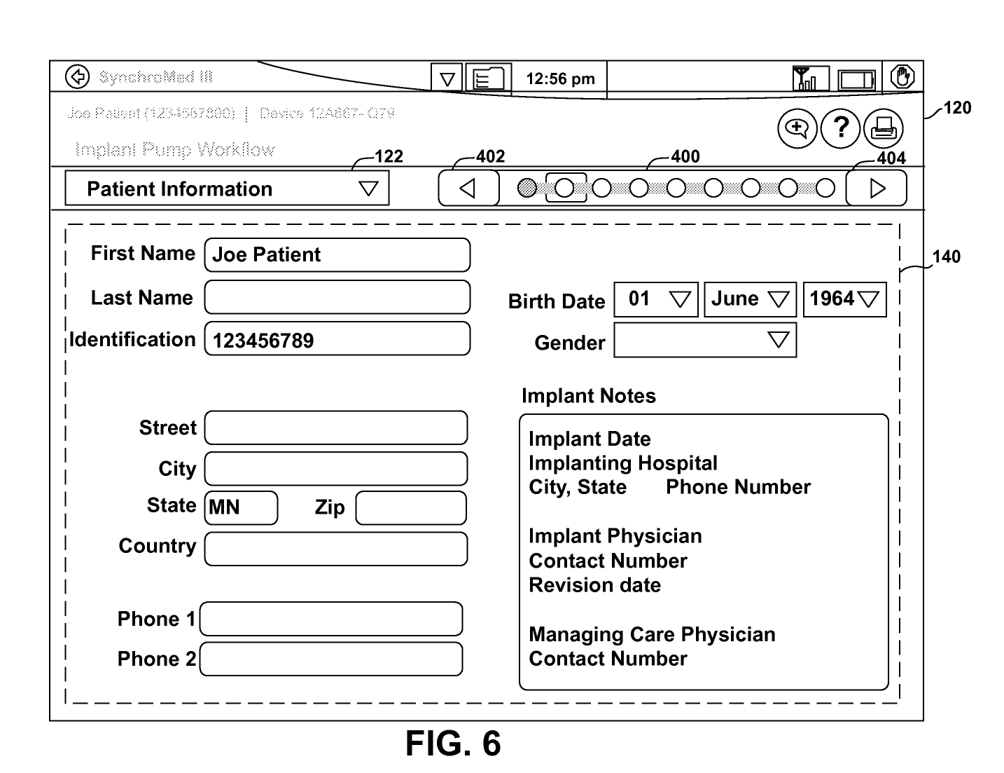

FIG. 6 is a screenshot illustrating an example user interface screen 120 for editing patient information 140. User interface screen 120 displays patient information 140 when a user selects “patient information” from drop-down menu 122. The user may fill in fields of patient information 140 to save patient data to memory 86 of programmer 20, such that the patient information can be retrieved at a later time. This information may also assist the user in selecting a therapy regimen for patient 16. As shown in FIG. 6, the patient information 140 may include a variety of information, such as name, gender, address, identification number, and birth date, as well as various implant notes that may be recorded by a clinician with respect to the implant date, site, and physician for a particular patient.

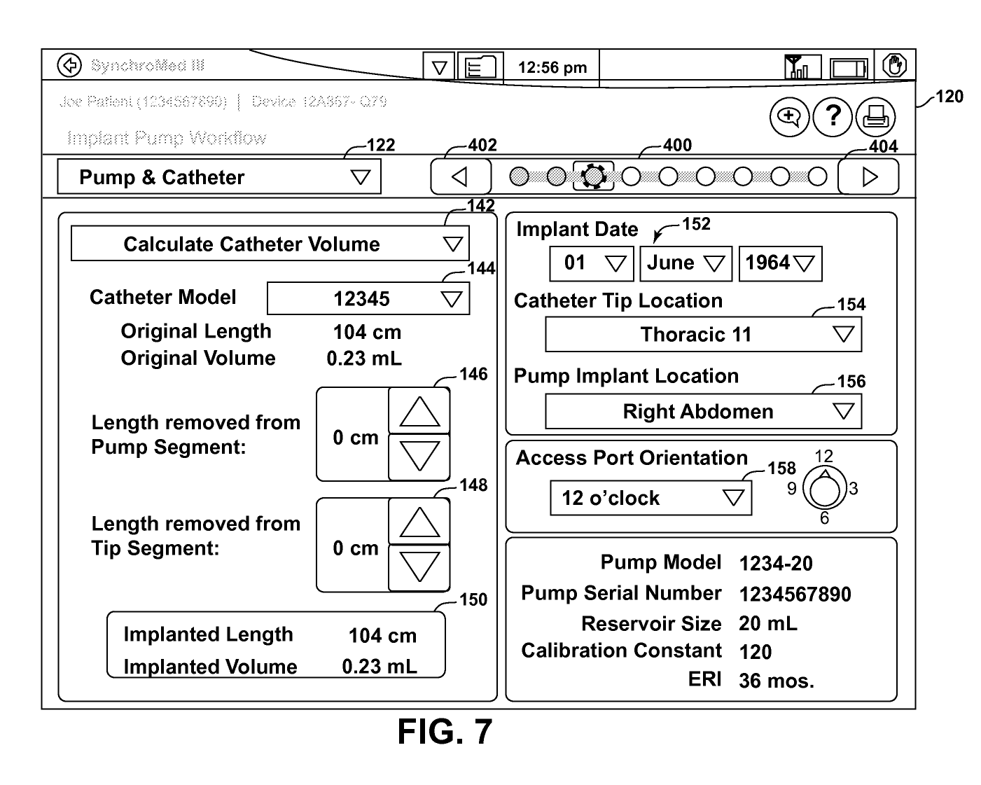

FIG. 7 is a screenshot illustrating an example user interface screen 120 for editing drug pump and catheter information. User interface screen 120 displays information for editing drug pump and catheter information when the user selects “pump and catheter” from drop-down menu 122. For example, user interface screen 120 enables a user to describe catheter volume, a date of implantation of IMD 12, a location of IMD 12, and other information regarding IMD 12.

The user can select various methods for entering catheter volume from catheter volume drop-down menu 142. In the example of FIG. 7, the user has selected “calculate catheter volume,” which causes programmer 20 to calculate the catheter volume from various data regarding the catheter. In another example, the user may select “enter catheter volume” from drop-down menu 142, e.g., when the user knows the volume of the catheter and desires to enter the volume directly, rather than having programmer 20 calculate the volume. In the example of FIG. 7, the user selects a catheter model from catheter model drop-down menu 144. Programmer 20 stores default values for potential catheter models for the catheter's length and volume before the catheter has been modified.

When drug pump IMDs are implanted in patients, surgeons commonly remove one or more segments from the catheter of the IMD. To determine a proper bolus amount for priming, the surgeon who removed this segment from the IMD must record the length of the segment removed. The user of programmer 20 may therefore enter the length of the segment removed from the catheter using user interface screen 120 by selecting the length removed from the pump segment with arrows 146 and the length removed from the tip segment with arrows 148. When user interface screen 120 receives removed-length data from arrows 146 and 148, user interface screen 120 sends the data to processor 84, which calculates a resulting length for the catheter, as well as a resulting volume for the catheter, e.g., in accordance with a program or module stored in memory 86. The processor then returns the results to be displayed to user interface screen 120, and user interface screen 120 displays the resulting length and the resulting volume for the catheter in output box 150.

User interface screen 120 also allows a user to enter a date that IMD 12 was implanted with boxes 152. The user also enters a location where the tip of catheter 18 of IMD 12 was implanted using drop-down menu 154, as well as a location of IMD 12 itself using drop-down menu 156. In the example of FIG. 7, the catheter tip location is indicated as “Thoracic 11,” and the pump implant location is indicated as “right abdomen.” A subsequent user of programmer 20 may therefore determine the location of IMD 12, e.g., to refill reservoir 30 of IMD 12 with fluid. The user also enters the orientation of the access port using drop-down menu 158. The orientation of the access port assists a user in determining the location and orientation of refill port 26 of IMD 12. Various pump information such as pump model, serial number, reservoir size, calibration constant and elective replacement indicator (ERI) may be presented on the screen of FIG. 7.

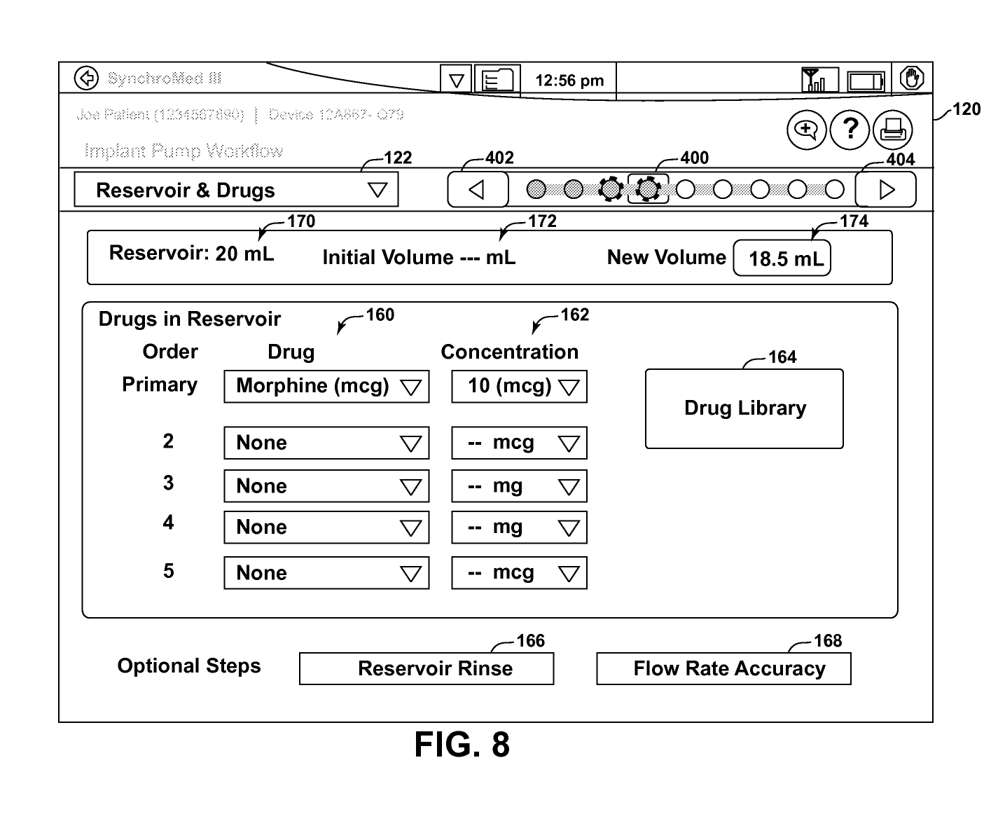

FIG. 8 is a screenshot illustrating an example user interface screen 120 for editing drug information regarding a drug delivered by IMD 12. User interface screen 120 enables a user to modify drug information for drugs delivered by IMD 12. Therefore, when a subsequent user interrogates IMD 12, the subsequent user may determine the information regarding the drug delivered by IMD 12.

User interface screen 120 depicts reservoir information that indicates a maximum volume 170 of reservoir 30 of IMD 12, a starting or initial volume 172 of drug within reservoir 30 as of the time of the programming session, and a new volume 174 that will be inserted into reservoir 30 after the programming session has been completed. In one example, IMD 12, programmer 20, or both, determine initial volume 172 at the beginning of a programming session by subtracting the dispensed volume of drug delivered to patient 16 since the last programming session from the new volume 174 entered at the time of the last programming session. The dispensed volume can be determined by multiplying the rate of drug delivery (volume/time) by the amount of elapsed time drug was dispensed at that rate.

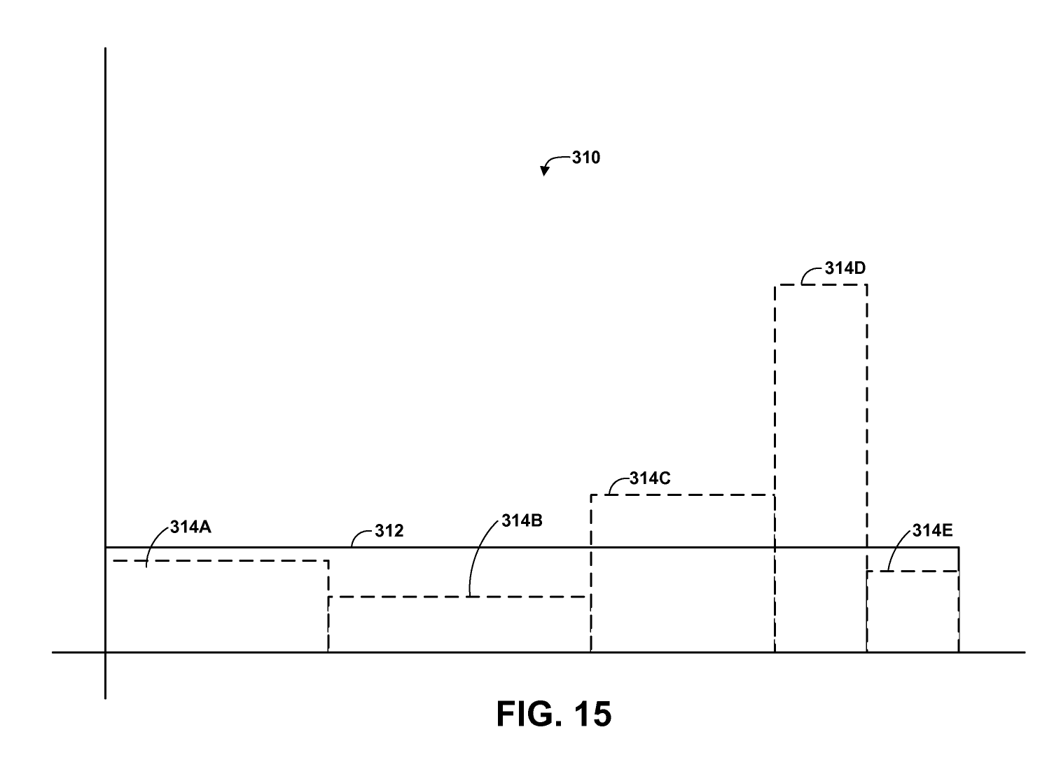

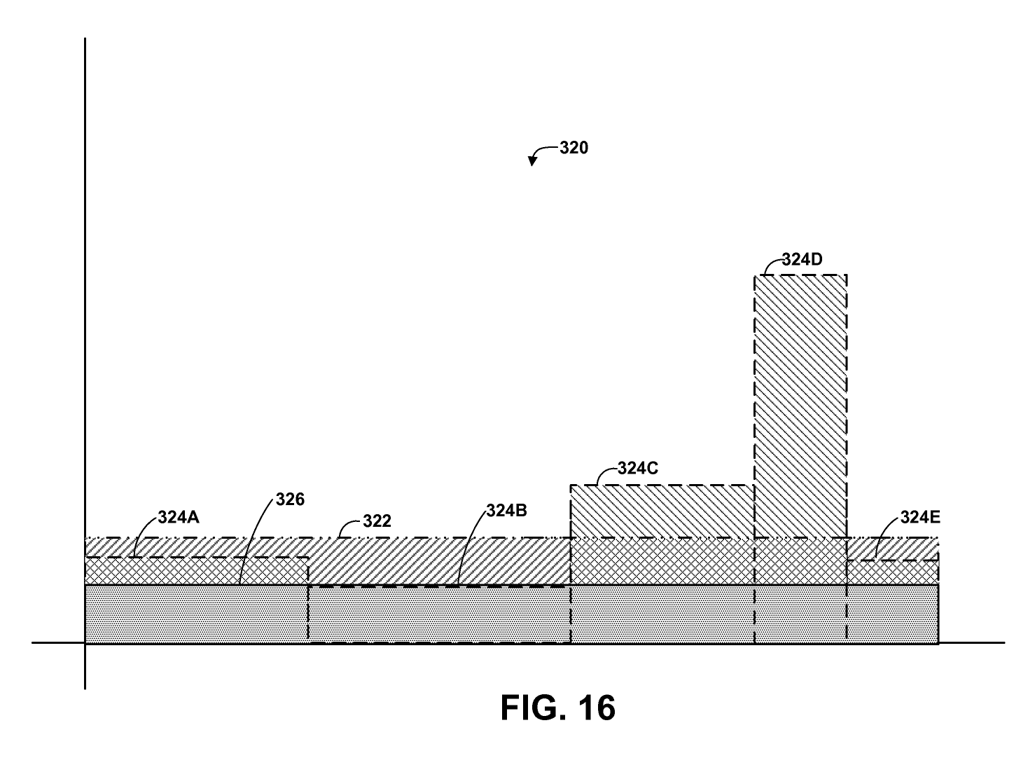

The user may also enter drug information into drug selection area 160 and concentration selection area 162. The user may select a drug from a drop-down menu of drug selection area 160 that is to be delivered to reservoir 30 of IMD 12. The user may also select a corresponding concentration of the drug from a corresponding drop-down menu of concentration selection area 162. Example drugs that may be listed by drop-down menus of drug selection area 160 include saline (as a default filler or as a placebo during clinical testing or other drug trails), morphine, bupivacaine, clonidine, hydromorphone, baclofen, alpha adrenergic agonists, baclofen, fentanyl, sufentanil, ziconotide, or other drugs or fluids. Selecting drugs from multiple drop-down menus of drug selection area 160 indicates that a combination of drugs is to be delivered to reservoir 30. For example, a user may select “morphine” from a first drop-down menu and “bupivacaine” from a second drop-down menu of drug selection area 160. When IMD 12 has already been programmed according to a dosing program with a first fluid, i.e., a fluid containing a drug at a particular concentration, user interface screen 120 displays the first drug and the first concentration of the drug. If the user changes the drug, e.g., as discussed with respect to the example of FIG. 15, processor 84 may recognize the fact that the drug and/or concentration has changed and may cause user interface screen 120 to suggest that a bridge be performed, e.g., as discussed with respect to FIG. 16.

The user may also select Drug Library 164 to link to a drug library. User interface screen 120 may then display a list of drugs and information regarding each of the listed drugs, such as names of the drugs, one or more common concentration values, and units for the values. In one example, the user may select a drug from the drug library, including a concentration of the drug, and user interface screen 120 populates one or more of drop-down menus 160 and/or 162 with the user's selection. In one example, the drug library may be user definable, allowing a user to enter names of drugs, one or more concentrations that are used for the drugs, and units for the concentrations. The user may also perform optional steps during the programming session, such as performing a reservoir rinse to rinse reservoir 30 of IMD 12 by selecting reservoir rinse button 166 or determining flow rate accuracy of IMD 12 by selecting flow rate accuracy button 168.

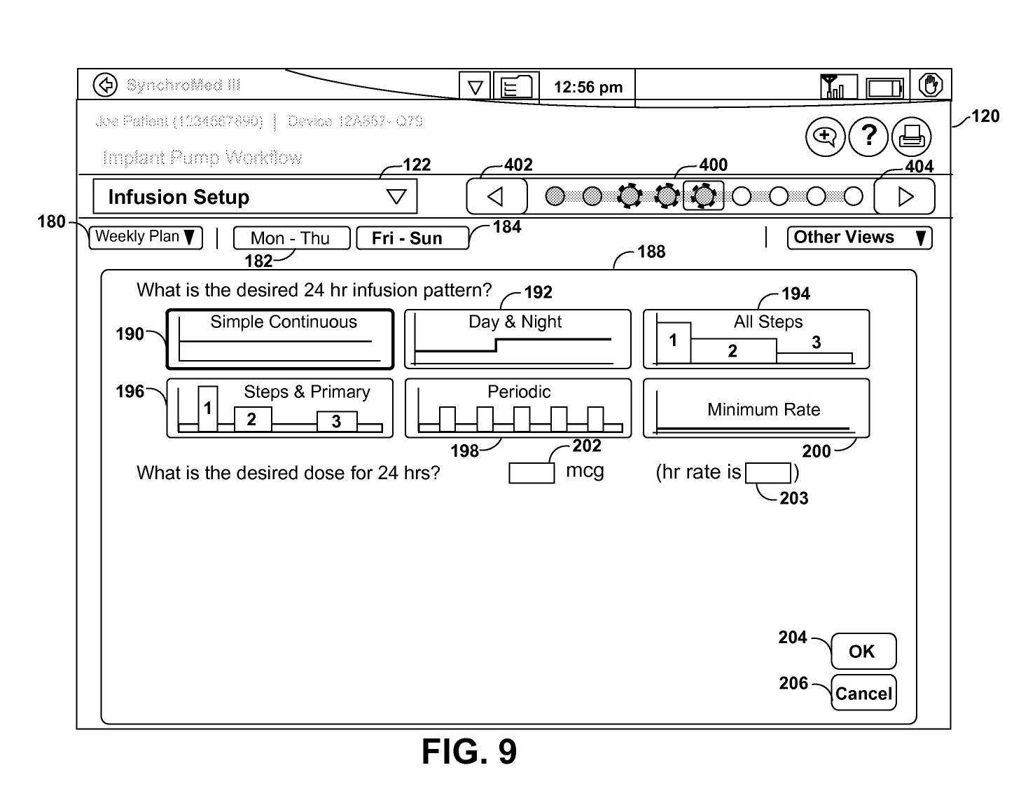

FIG. 9 is a screenshot illustrating an example user interface for selecting, programming, and/or modifying a therapy schedule of a dosing program. User interface screen 120 presents window 188, which allows a user to program one or more therapy schedules for one or more pending dosing programs. Window 188 may also enable the user to modify an existing or a current dosing program, e.g., one or more therapy schedules of the current dosing program. For example, window 188 includes Simple Continuous button 190 that allows the user to modify a simple, continuous therapy schedule, i.e., a therapy schedule according to which IMD 12 will deliver a programmed dose at a constant infusion rate at all times. Day & Night button 192 allows the user to customize a first rate of fluid delivery for a daytime period and a second rate of fluid delivery for a nighttime period. All Steps button 194 allows the user to modify a plurality of rates, enabling the user to select how many different “steps” are desired and modifying each step, e.g., as described with respect to FIG. 12. Steps+Primary button 196 allows the user to modify a periodic therapy schedule where various doses are delivered at different rates, with periods of time during which no fluid is delivered. Periodic button 198 allows the user to establish a periodic dose at fixed intervals, where each dose may include the same quantity of fluid delivered and the same time period over which the fluid is delivered. Minimum Rate button 200 allows the user to program IMD 12 to continuously deliver the lowest dose of fluid possible.

User interface screen 120 may also allow a user to establish two or more different therapy schedules for different time periods. For example, the user may establish a first therapy schedule for days Monday to Thursday of a week, and a second therapy schedule for days Friday to Sunday of the week, by selecting Mon-Thu button 182 to establish the first therapy schedule and Fri-Sun button 184 to establish the second therapy schedule, in the example of FIG. 9. This disclosure may refer to the second therapy schedule as an alternative therapy schedule. The user may also modify the number of different periods, as well as which days belong to each period, through Weekly Plan drop-down menu 180. For example, the user may select “Daily,” “Mon-Thu and Fri-Sun,” “Mon-Fri and Sat-Sun,” “Mon, Tues, Wed, Thu, Fri, Sat, Sun,” or other options from Weekly Plan drop-down menu 180. User interface screen 120, accordingly, enables the user to modify the therapy schedule of the dosing program for each of the periods the user selects from Weekly Plan drop-down menu 180. For example, when the user selects “Mon, Tues, Wed, Thu, Fri, Sat, Sun,” user interface screen 120 enables the user to create and/or modify a therapy schedule for each day of the week. As another example, when the user selects “Mon-Thu and Fri-Sun”, user interface screen 120 enables the user to create a first therapy schedule for days Monday-Thursday and a second therapy schedule for days Friday-Sunday. The user may enter a total dose for a 24-hour period in text box 202 as a number in micrograms or an average hourly rate in text box 203 as a number corresponding to an amount of fluid to be delivered per hour.

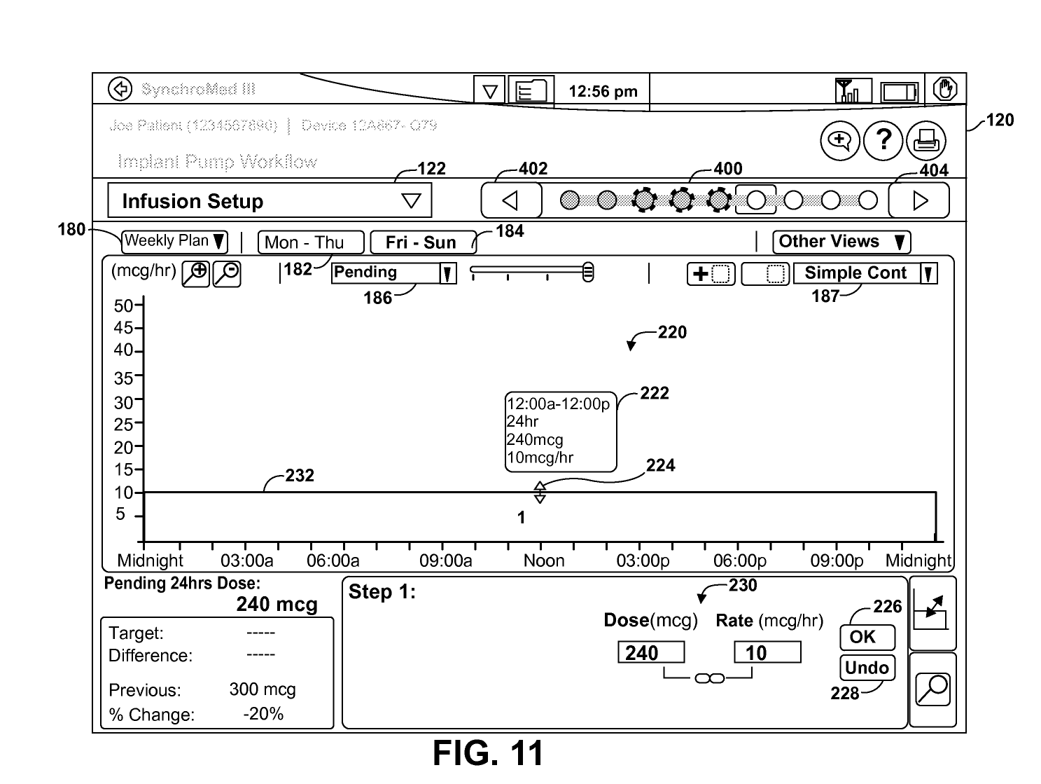

After the user has selected a therapy schedule from window 188 and entered values in either text box 202 or text box 203, the user may select OK button 204 to modify or customize the therapy schedule. Processor 84, upon receiving an indication that the user has selected OK button 204 via user interface screen 120, causes user interface screen 120 to display a user interface to modify or customize the dosing program, e.g., one of the user interfaces of FIG. 11 or FIG. 12. Processor 84 may execute instructions stored in memory 86 to control user interface screen 120. The user may also select Cancel button 206, in response to which processor 84 causes user interface screen 120 to return to a previous screen, e.g., one of the screens of FIGS. 4-8.

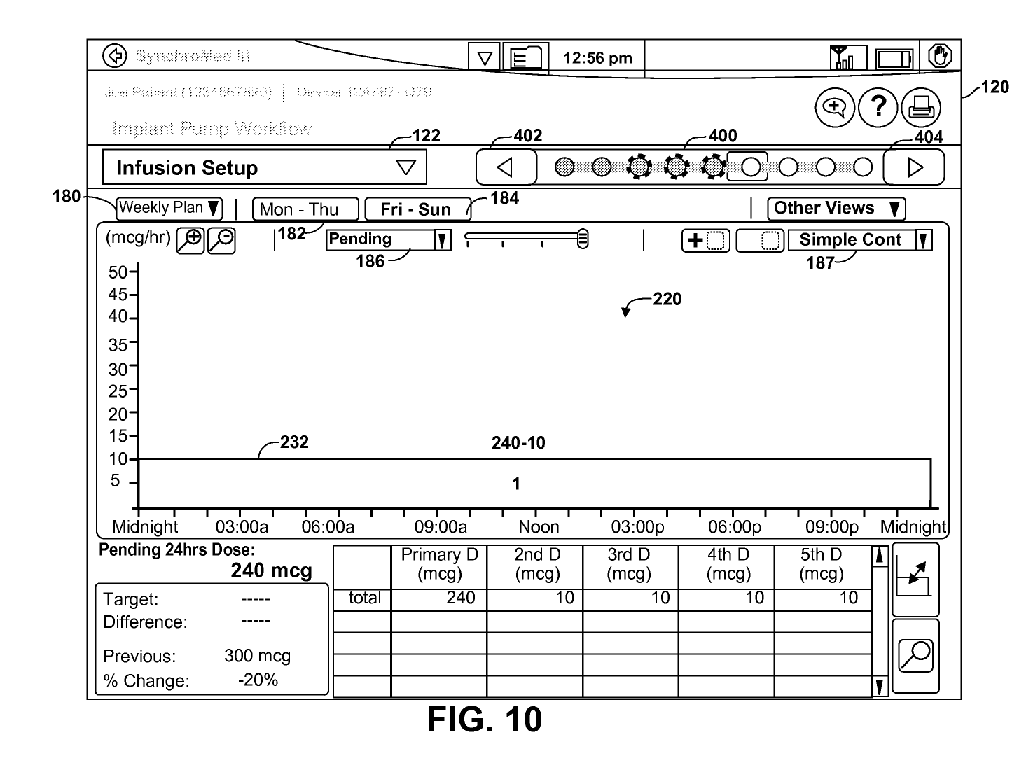

FIG. 10 is a screenshot illustrating an example user interface that displays a therapy schedule based on user input received via the user interface of FIG. 9. Graph 220 provides a graphical representation of a pending dosing program that includes the therapy schedule. The dosing program may be pending in that the dosing program has not yet been downloaded to IMD 12 via telemetry module 88 of programmer 20 or has been downloaded to IMD 12 but has not yet been selected or activated to control operation of IMD 12. In either case, the clinician may be evaluating the pending program. When IMD 12 is operating according to a dosing program stored in memory 40 of IMD 12, the dosing program according to which IMD 12 is operating may be referred to by this disclosure as a current dosing program. When a dosing program has not been downloaded and stored in memory 40 of IMD 12, or has been downloaded but IMD 12 is not operating according to the dosing program, the dosing program also may be referred to as pending in this disclosure. That is, the dosing program in memory 40 may not have been activated by programmer 20, and therefore the dosing program remains pending.

In the example of FIG. 10, graph 220 includes a bar chart, with step 232 at 10 mcg/hr for the entire 24 hour period. Programmer 20 constructs graph 220 based on information provided by the user with the user interface of FIG. 9 after the user selects OK button 204. That is, example graph 220 is based on a selection from the user of a simple, continuous graph, using Simple Continuous button 190, and entering “240” in text box 202, then selecting OK button 204. Graph 220 also includes textual representation 221 of “240-10,” which indicates that 240 mcg are to be delivered to the patient by IMD 12 for the 24 hour period, with 10 mcg to be delivered per hour for each hour of the 24 hour period.

FIG. 11 is a screenshot illustrating an example user interface that allows a user to modify a therapy schedule of a pending dosing program. The user interface of FIG. 11 may also be used to modify a therapy schedule of a current dosing program to create a new pending dosing program. User interface screen 120 allows the user to modify the therapy schedule by manipulating graph 220. After the user sees the therapy schedule based on the default input provided by processor 84 to window 188 (FIG. 9), the user may desire to modify the therapy schedule generated by programmer 20. To modify the therapy schedule, the user may select arrows 224 to drag step 232 up or down. A movement either up or down along the vertical axis corresponds to an increase or decrease in the rate at which fluid/drug will be delivered to patient 16 by IMD 12.

Processor 84 may be configured to cause user interface screen 120 to present balloon text box 222 as the user moves arrows 224 up or down to indicate the starting and ending times corresponding to step 232, the total time represented by step 232, the total dose administered during the time of step 232, and the rate of fluid delivery corresponding to step 232. User interface screen 120 also illustrates the total dose for the 24-hour period represented by graph 220 as well as the average rate for the 24-hour period with dose-and-rate information 230. In this example, the total dose and average rate shown in balloon 222 and dose-and-rate information 230 are 240 mcg and 10 mcg/hour.

After the user has modified step 232 to program IMD 12, the user may select OK button 226. When the user wishes to undo one or more movements of step 232, the user may select Undo button 228. In response to each activation of the Undo button 228, processor 84 may undo a previous modification. In some cases, the clinician may step back through several actions using the Undo button 228. The user may also change the therapy schedule by selecting therapy schedule drop-down menu 187, which may list options such as “simple continuous,” “all steps,” “day & night,” “steps+primary,” “periodic,” and “minimum rate,” or other options. Upon selection of a different therapy schedule from drop-down menu, the user interface presents a corresponding therapy schedule for viewing and/or modification by the clinician.

FIG. 12 is a screenshot illustrating another example user interface by which a user may modify a therapy schedule. The example therapy schedule of FIG. 12 is an “All Steps” therapy schedule, as indicated by drop-down menu 187. An all-steps therapy schedule includes a plurality of steps, such as steps 234A-234D (steps 234), each of which has a time and a dose. The time length of the step corresponds to the width of the step, and the dose of the step corresponds to the height of the step. The time at which the step is delivered corresponds to the position of the step along the time axis. A clinician may adjust the therapy schedule by adjusting the widths and heights of individual steps, and repositioning the steps along the time line.

In the example of FIG. 12, step 234A (step 1) has a height corresponding to 22 mcg/hour and a width corresponding to 3 hours and 38 minutes. Step 234B (step 2) has a height corresponding to 12.5 mcg/hour and a width corresponding to 9 hours and 12 minutes. Step 234C (step 3) has a height corresponding to 32 mcg/hour and a width corresponding to 2 hours and 45 minutes. Step 234D (step 4) is illustrated with a dashed line outline to indicate that step 234D has not yet been configured. Steps 234A-D span a 24-hour period in the example of FIG. 12. In other examples, different periods of time may be used.

For example, if the left side arrow 235 of step 234C is moved to the right, repositioning the start of the step 234C to a later time, then the end at the right edge of step 234B may extend to the right, lengthening the overall time of step 234B. If the left side arrow 235 of step 234C is moved to the left, however, repositioning the start of the step 234C to an earlier time, then the end at the right edge of step 234B may extend to the left, shortening the overall time of step 234B. Hence, the left and right edges of each step may be considered start and end times of the respective steps. The start and end times determine the times within the 24 hour period that delivery of fluid according to a step starts and ends, respectively. The length of the step between the start and end times determines the length of time for which fluid is delivered according to a step.

As illustrated in the example of FIG. 12, the user is in the process of editing step 234C by dragging one or more of arrows 235 either up, down, left, or right, e.g., with a stylus or other pointing device applied to a touch screen or non-touch screen. By dragging arrows 235, the user may change the programmed dose of the therapy schedule corresponding to step 234C. For example, the user may drag one of arrows 235 to the right or left to increase or decrease the time during which the rate is to be delivered. The user may also drag one of arrows 235 up or down to increase or decrease the rate for step 234C. Upon moving one of arrows 235 to the right or left to adjust the size of a given step, such as step 234C, adjacent steps may automatically adjust in response to the resizing of step 234C. The user may also drag a step to a different location without resizing the step, which may cause user interface screen 120 to automatically resize steps on either side of the relocated step. In one example, a user may cut-and-paste a step, reorder steps, copy steps, paste steps, or otherwise move or adjust the steps, or add or remove steps.

User interface screen 120 may also provide summary information regarding the pending dosing program. For example, in FIG. 12, user interface screen 120 displays window 236, which illustrates the total dose of a drug that will be delivered according to programmed steps 234 over the 24 hours period according to the pending dosing program. Window 236 indicates that, in the example of FIG. 12, the 24-hour dosing program will deliver 275 mcg of drug to patient 16. Window 236 also illustrates that the target dose for the 24-hour period is 400 mcg, which may have been previously set by the clinician. In addition, window 236 may indicate the difference between the target and the programmed 24-hour doses (e.g., 125 mcg in FIG. 12). Window 236 further displays an initial 24-hour dose, representing the 24-hour dose of a previous dosing program, e.g., a current dosing program.

User interface screen 120 may also display step-specific information 238. In the example of FIG. 12, step-specific information 238 corresponds to step 234C. Step-specific information includes start-and-end time text field 240, which corresponds to the width of step 234C. Step-specific information 238 may present information for a particular step that is selected, e.g., “clicked on,” by the clinician. Dose text field 242 corresponds to a calculation by processor 84 of the total quantity of drug to be delivered to patient 16 during step 234C by IMD 12. Rate text field 244 depicts the rate at which drug is to be delivered to the patient during step 234C, corresponding to the height of step 234C. In one example, the user may enter data in text fields 240-244 and the corresponding step, e.g., step 234C, will change shape automatically, without the user needing to move or resize the step with arrows 235. The user may select OK button 248 to accept a therapy schedule of a pending dosing program or Cancel button 246 to cancel programming of the therapy schedule and return to a previous screen. In one example, user interface screen 120 may present a button, window, field, menu, or other means that enable the user to add a step or delete a step for a therapy schedule of a pending dosing program.

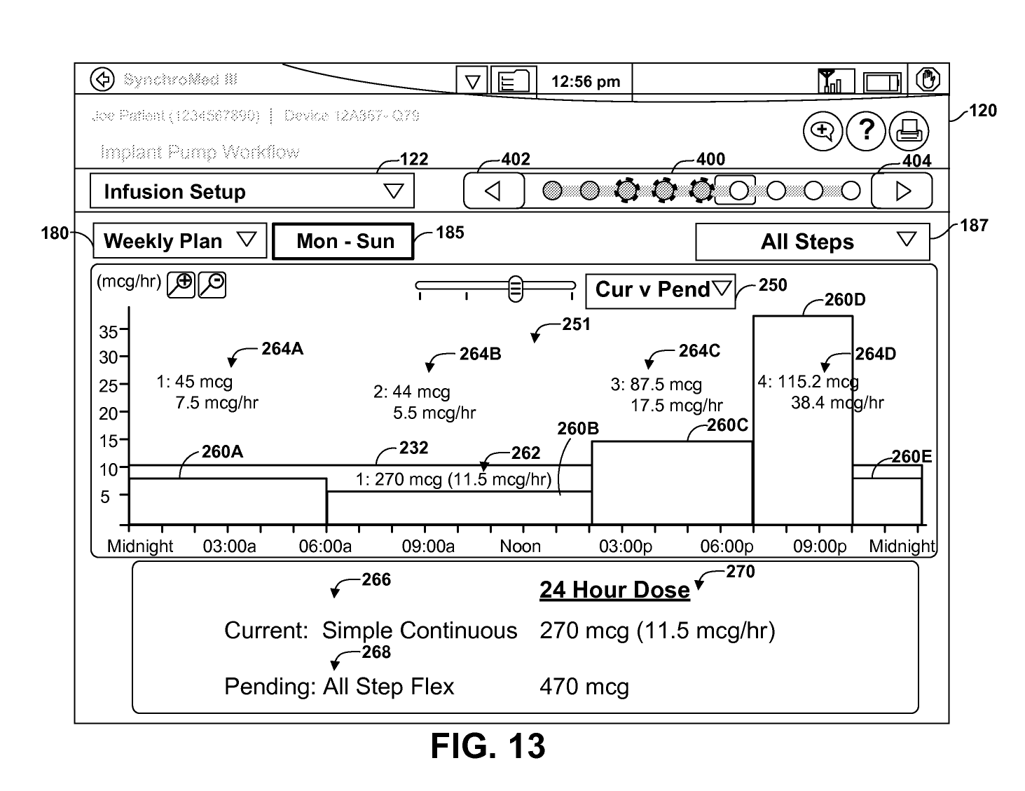

FIG. 13 is a screenshot illustrating an example user interface for displaying a therapy schedule of a current dosing program simultaneously with a therapy schedule of a pending dosing program. That is, processor 84 may cause user interface screen 120 to display a representation, such as the example of FIG. 13, of a current dosing program, i.e., a dosing program according to which IMD 12 is currently administering a therapy, and simultaneously display a representation of a pending dosing program, i.e., a dosing program according to which IMD 12 is not currently administering a therapy, e.g., either because the dosing program has not yet been downloaded to IMD 12 or because the dosing program has not been activated by programmer 20 though the dosing program may reside in memory 86 of IMD 12. In particular, in the example of FIG. 13, step 232 corresponds to a current dosing program while steps 260A-260E (steps 260) correspond to a pending dosing program. Step 232 provides an indication of the current dosage program, while steps 260 represent a therapy schedule of the pending dosing program. In one example, steps 260 may correspond to steps 234 (FIG. 12) after the user has finished manipulating steps 234 to program the therapy schedule of the pending dosing program.

To display graph 251, which includes step 232 and steps 260, the user may select “Cur v Pend” (current vs. pending) from display selection drop-down menu 250. Processor 84 then causes user interface screen 120 to display, e.g., a graphical representation of a current dosing program and a graphical representation of a pending dosing program simultaneously. Drop-down menu 250 may include other options, such as “current” to display a representation of the current dosing program or a therapy schedule of the current dosing program of IMD 12, and “pending” to display the pending, i.e., newly programmed, dosing program or a therapy schedule of the pending dosing program. Selecting “pending” from drop-down menu 250 may also enable the user to modify the infusion pattern or to create a new infusion pattern. In one example, the user may retrieve a current dosing program of IMD 12, modify the current dosing program, and then download the modified dosing program to IMD 12. The modified current dosing program may therefore be considered a “pending” dosing program, and IMD 12 may continue to operate according to the unmodified current dosing program until programmer 20 downloads or otherwise activates the modified dosing program to IMD 12.

Graph 251 may provide graphical representations of both the current dosing program (i.e., step 232) and the pending dosing program (i.e., steps 260). In addition, user interface screen 120 may provide textual representations of the dosing programs. For example, user interface screen 120 illustrates textual representation 262, corresponding to step 232. User interface screen 120 also illustrates textual representations 264A-264D, corresponding to steps 260A-260D. User interface screen 120 also may provide textual representations of the style of infusion pattern for each of the dosing programs: text representation 266 (“Simple Continuous”) for the current (i.e., initial) dosing program and text representation 268 (“All Step Flex”) for the pending dosing program. User interface screen 120 also may provide summary textual representation 270, which summarizes differences between the current dosing program and the pending dosing program. In the example of FIG. 13, summary textual representation shows that for the 24-hour dosage period, the current dosing program provides 270 mcg of drug at 11.5 mcg/hour, while the pending dosing program will provide 470 mcg of drug.

User interface screen 120 may represent differences between the current dosing program and the pending dosing program in a variety of ways. For example, user interface screen 120 may illustrate the current dosing program (e.g., step 232) with a first color and the second dosing program (e.g., steps 260) with a second color. Other distinguishing features may include line styles, shading, hatching, or other means for distinguishing two graphs. In one example, processor 84 may calculate a difference between a rate represented by a first step of a first dosing program and a rate represented by a second step of a second dosing program, where both the first step and the second step occur at the same temporal location, and user interface screen 120 may display the calculated difference as an absolute difference, as a percentage difference, with a colored band (e.g., green for increase and red for decrease, different colors to represent different percentage changes, etc.), arrows to represent increase or decrease, varying line thickness, or other visual queues to represent the difference.

In this manner, FIG. 13 illustrates an example screenshot for displaying, with a device for programming an implantable fluid delivery device, a first indication of a selected dosing program of the implantable fluid delivery device and displaying, simultaneously with the first indication, a second indication of a pending dosing program of the implantable fluid delivery device.

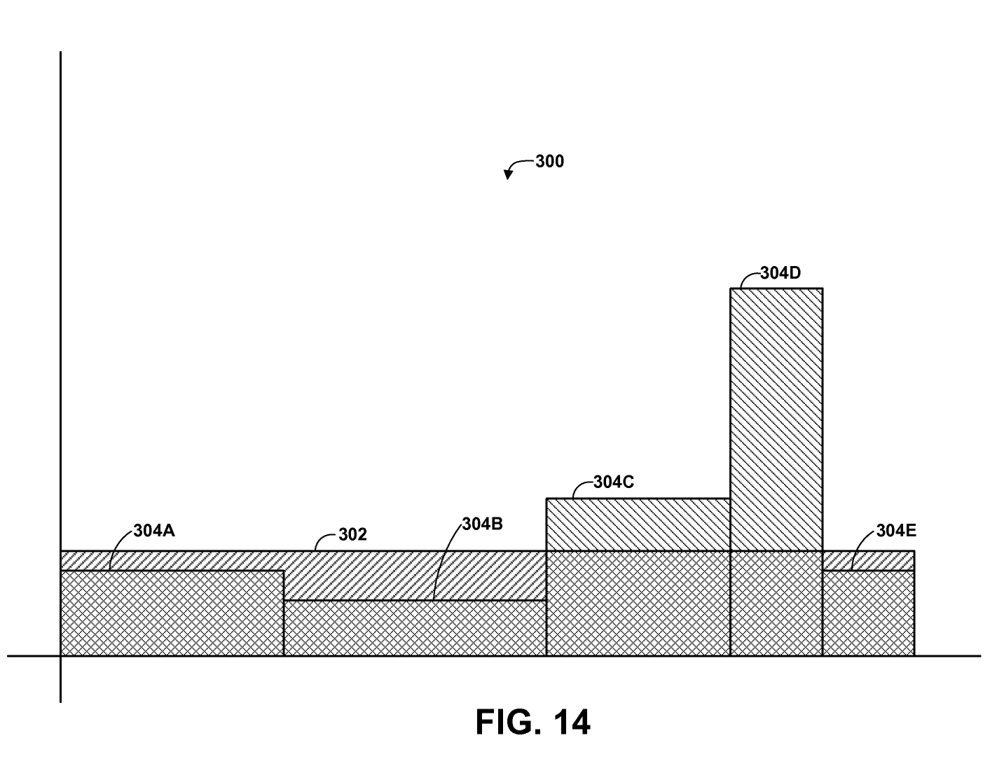

FIG. 14, for example, is a block diagram illustrating graph 300, which may correspond to graph 251, which graphically represents various dosing programs with distinct shading styles. Step 302 of FIG. 14 may correspond to step 232, while steps 304A-304E (steps 304) may correspond to steps 260. Step 302 is shaded in FIG. 14 with left-to-right slanted lines, while steps 304 are shaded with right-to-left slanted lines. Where steps 304 overlap step 302, both left-to-right slanted lines and right-to-left slanted lines appear.

FIG. 15, as another example, is a block diagram illustrating graph 310, which may correspond to graph 251, which graphically represents various dosing programs with distinct line styles. Step 312 of FIG. 15 may correspond to step 232, while steps 314A-314E (steps 314) may correspond to steps 260. Step 312 is displayed in FIG. 15 with solid lines, while steps 314 are displayed with dashed lines.

FIG. 16 is a block diagram that presents another example graph 320 that displays three graphical representations of three distinct dosing programs. Step 322 is a graphical representation of an example current dosing program, which in the example of FIG. 16 is a continuous dosing program. Steps 324A-324D are graphical representations of steps of an example pending dosing program, which in the example of FIG. 16 is an all-steps dosing program. Step 326 is a graphical representation of an example third dosing program. Step 326 may comprise a representation of a historical dosing program, i.e., a dosing program that was administered to patient 16 by IMD 12, or another IMD implanted within patient 16, at some time prior to the current dosing program. In general, any of a pending dosing program, current dosing program, historical dosing program, and alternative dosing program may be a continuous dosing program (according to which IMD 12 administers fluid at a constant rate at all times), an all-step dosing program (where a user defines rates and temporal durations for each of one or more steps of the dosing program), a day-night dosing program (according to which IMD 12 administers fluid at a first rate during the day and a second rate during the night), a periodic dosing program (according to which IMD 12 administers fluid at a defined rate for a defined time for each of a plurality of dosing periods, between which IMD 12 does not administer fluid or administers fluid at a minimum rate, which may correspond to a non-therapeutic rate) or other variety of dosing program.

Step 326 may, in another example, correspond to a representation of a first alternative therapy schedule of a pending dosing program, while steps 324 may represent a second alternative therapy schedule of the pending dosing program. That is, the pending dosing program may define two alternative therapy schedules, each of which includes respective programmed doses. For example, as illustrated in FIG. 9, the user may select Mon-Thu button 182 or Fri-Sun button 184 from user interface screen 120. The user may program a first therapy schedule for the pending dosing program for days Monday to Thursday and a second therapy schedule, as an alternative to the first therapy schedule, for days Friday to Sunday. IMD 12, upon receiving the pending dosing program and an indication that IMD 12 is to deliver fluid according to the pending dosing program (e.g., an activation of the pending dosing program), may deliver fluid to patient 16 according to the first therapy schedule Monday through Thursday, then deliver fluid to patient 16 according to the second (i.e., alternative) therapy schedule Friday through Sunday.

Step 322 is illustrated with both left-to-right slanted lines for shading and a double-dotted-dashed line style. Steps 324 are illustrated with right-to-left slanted lines for shading and a dashed line style. Step 326 is illustrated with spotted shading and a solid line style. Other examples may display additional graphical representations for any number of infusion patterns. Each of the infusion patterns for various dosing programs may be displayed using variations in color, shading, line styles, or other differentiating features.

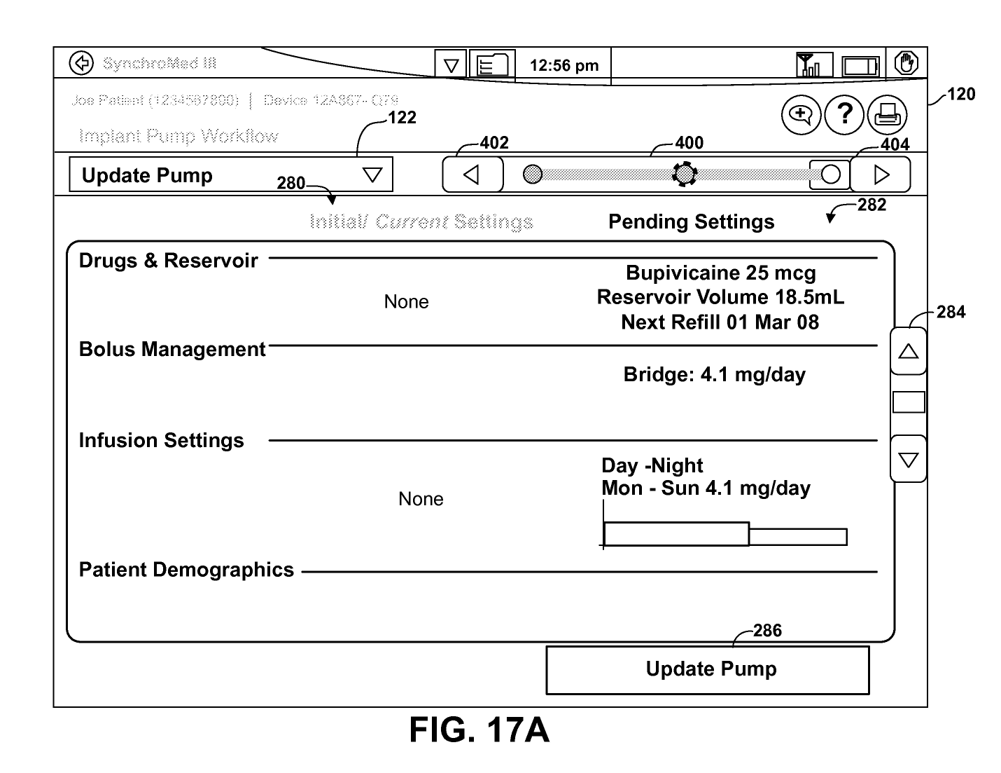

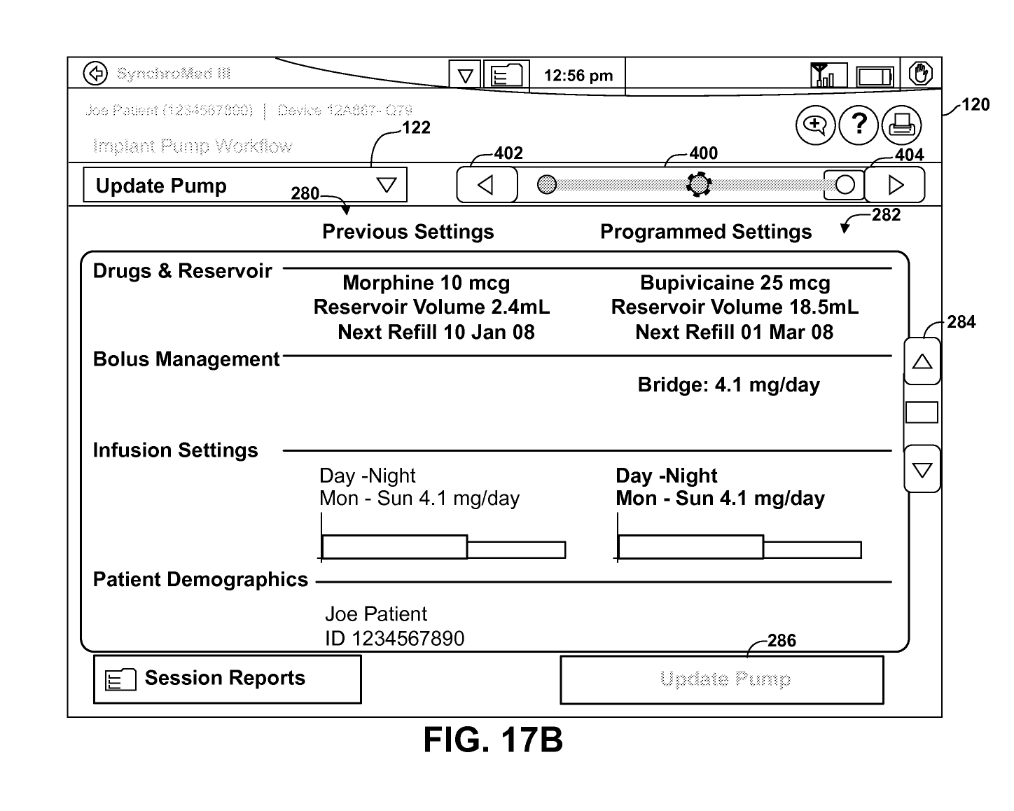

FIG. 17A is a screenshot of an example user interface that displays current settings of IMD 12 simultaneously with pending settings of IMD 12. User interface screen 120 may present the example user interface of FIG. 17 when the user selects “Update Pump” from drop-down menu 122. The example of FIG. 17A depicts initial/current settings 280 for IMD 12 before IMD 12 has ever been programmed, and pending settings 282 for the first dosing program sent to IMD 12. In the example of FIG. 17A, various settings are indicated as “None” in the event the IMD 12 has not yet been programmed. However, an interface similar to that of FIG. 17A may also be displayed to show differences between a current dosing program and a pending dosing program, e.g., as discussed with respect to FIG. 17B. A user of programmer 20 may scroll through various settings using scroll buttons 284. In one example, user interface screen 120 enables the user to modify pending settings 282 through the user interface presented in FIG. 17A. In another example, the user may switch to another screen by selecting the screen to be modified from drop-down menu 122.

FIG. 17B is a screenshot of another example user interface that displays current settings of IMD 12 simultaneously with pending settings of IMD 12. The example of FIG. 17B depicts previous settings 280 for a current dosing program of IMD 12 after IMD 12 has already been programmed for a patient and programmed settings for IMD 12 for a pending dosing program. Previous settings 280 in the example of FIG. 17B illustrate that a therapy schedule of current dosing program is set as a day/night dosing program, i.e., IMD 12 delivers 4.9 mg of a drug per day with a first rate for the daytime and a second rate for the nighttime. Programmed settings 282 reveal, however, that IMD 12 will deliver 4.1 mg of drug per day according to a different day/night dosing program that is being considered as a pending program for operation of IMD 12.

FIGS. 17A and 17B depict example screenshots for displaying, with a device for programming an implantable fluid delivery device, a first indication of a selected dosing program of the implantable fluid delivery device and displaying, simultaneously with the first indication, a second indication of a pending dosing program of the implantable fluid delivery device.

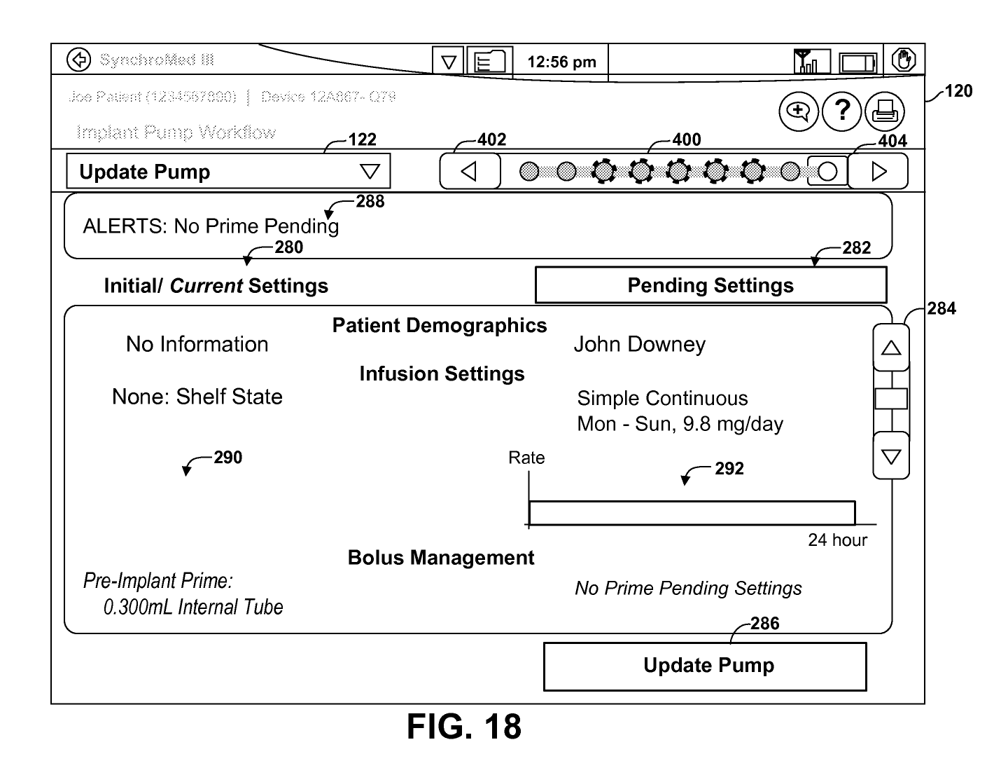

FIG. 18 is a screenshot of additional settings for both current settings 280 and pending settings 282 of IMD 12 that is displayed after the user has scrolled to a different set of information presented by user interface screen 120 with scroll buttons 284 for the example of FIG. 17A. FIG. 18 additionally includes a graphical representation of a current dosing program 290 and a pending dosing program 292. FIG. 18 also depicts alerts window 288, which informs a user of any alerts that the user may wish to address before programming IMD 12 with pending settings 282. Processor 84 of programmer 20 may cause user interface screen 120 to display an alert for any steps that processor 84 determines have not been performed by a user. For example, alerts window 288 depicts a message that the user has not yet programmed a priming phase of IMD 12. That is, processor 84 determines, according to instructions stored in memory 86, that the user should program a priming phase of IMD 12 before updating IMD 12 with the pending dosing program, e.g., by selecting Update Pump button 286.

When the user has verified that pending settings 282 are accurate, the user may select Update Pump button 286 to program IMD 12 with pending settings 282. When the user determines that pending settings 282 are not accurate, the user may select a window from drop-down menu 122 to modify pending settings 282. The user may also select left navigation arrow 402 to move to a previous screen and to edit the information of the screen. Updating IMD 12 by selecting Update Pump button 286 will program IMD 12 with pending settings 282, erasing all current settings 280. In one example, programmer 20 stores current settings 280 of IMD 12 in memory 86. In another example, programmer 20 interrogates IMD 12 to obtain the current settings from, e.g., memory 40 of IMD 12 via telemetry module 88 of programmer 20. For the example of FIG. 17A, user interface screen 120 of FIG. 18 may present further information for the previous and programmed settings operating to define current and pending dosing programs, respectively.

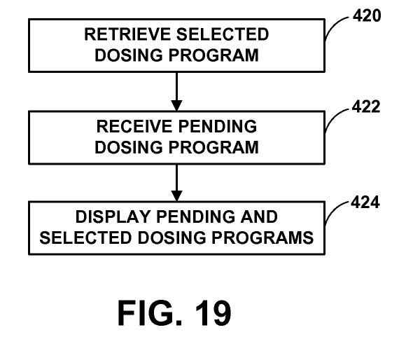

FIG. 19 is a flowchart illustrating an example method for displaying a first indication of a pending dosing program and a second indication of a selected dosing program, such as a current or a historical dosing program, simultaneously. Initially, programmer 20 retrieves a selected dosing program for IMD 12 (420). Programmer 20 may, in one example, retrieve the selected dosing program from memory 40 of IMD 12 via telemetry module 88. In another example, programmer 20 may retrieve the selected dosing program from memory 86 of programmer 20. The selected dosing program may be a current dosing program, historical dosing program, alternative dosing program, or other selected dosing program.

Programmer 20 then receives a pending dosing program for IMD 12 (422). The pending dosing program may be defined by a user via programmer 20, and evaluated in comparison to a selected dosing program. In some cases, a user may modify a selected dosing program to form the pending dosing program. The user may also input an entirely new dosing program as the pending dosing program. The pending dosing program may comprise a plurality of therapy schedules, each of which comprises one or more programmed doses, where each programmed dose defines a rate at which to deliver fluid and a time at which to deliver the fluid at the rate.

Programmer 20 then displays each of the selected dosing program and the pending dosing program, e.g., with user interface 82 (424). That is, programmer 20 may display a first indication of the selected dosing program of IMD 12 and a second indication of the pending dosing program of IMD 12. When the pending dosing program includes a plurality of therapy schedules, programmer 20 may display indications of each of the plurality of therapy schedules of the pending dosing program. Programmer 20 may display a first graph representative of the selected dosing program, wherein the first graph comprises a plurality of contiguous rectangles, each of the plurality of contiguous rectangles comprising a height representative of a rate of fluid delivery and a width representative of a time during which the dose is to be delivered, e.g., in a manner similar to that shown in FIG. 12. Each of the rectangles may be distinguished by a different shading style, line style, color, hatching, or other graphical or textual differentiation.

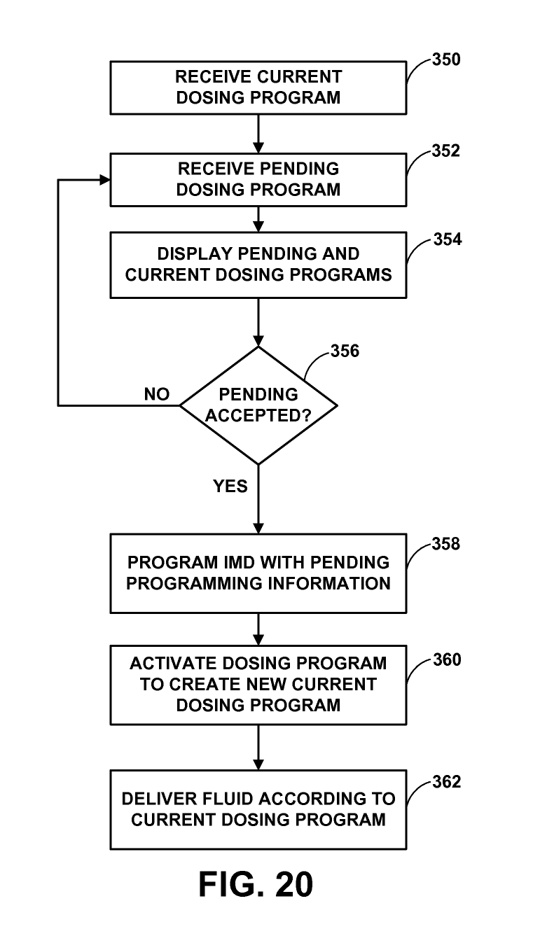

FIG. 20 is a flowchart illustrating an example method for displaying an indication of a first dosing program and an indication of a second dosing program simultaneously. In the example of FIG. 20, programmer 20 displays a current dosing program as the selected dosing program, as well as a pending dosing program, although in other examples, any of a current dosing program, a historical dosing program, an alternative dosing program, or other dosing program may be displayed as the selected dosing program. Initially, programmer 20 receives current programming information regarding the current dosing program of IMD 12 (350). Programmer 20 may retrieve the current dosing program from a memory, such as memory 86, or programmer 20 may interrogate IMD 12 to retrieve the current programming information from memory 40 of IMD 12. Programmer 20 may also receive the current dosing program in other ways, such as from communicating with another device that has stored the current dosing program.

Programmer 20 also receives a pending dosing program for IMD 12 (352). For example, programmer 20 may receive the pending dosing program through a user interface during a programming session conducted by the user. Programmer 20 may also receive the pending dosing program from another device that includes the pending dosing program, or programmer 20 may retrieve the pending dosing program from a memory, such as memory 86 or memory 40. Programmer 20 may, in some examples, receive modifications to an existing, e.g., a current dosing program of IMD 12, rather than an entirely new dosing program.

Programmer 20 then displays both the pending programming information and the current programming information via user interface 82 (354). In particular, programmer 20 displays a first indication of a first set of dosage information for a current dosing program of IMD 12 and, simultaneously, a second indication of a second set of dosage information for a pending dosing program of IMD 12. Programmer 20 may display the current and pending programming information textually, graphically, or both. For example, programmer 20 may display the current programming information using a first line style and the pending programming information using a second line style. Programmer 20 may also display the current programming information using a first shading style and the pending programming information using a second shading style. Programmer 20 may also display the current programming information using a first color and the pending programming information using a second color. As an example, programmer 20 may display graph 251, as discussed with respect to the example screenshot of FIG. 13.

Programmer 20 then waits for a user to accept the pending programming information (356). That is, programmer 20 waits for an indication that the user wishes to program IMD 12 according to the pending programming information. The user may cancel programming or modify the pending programming information (“NO” branch of 356), in which case programmer 20 receives new pending programming information (352).

When the user accepts the pending programming information (“YES” branch of 356), programmer 20 updates IMD 12 according to the pending dosing program (358). Programmer 20 may, for example, transmit the pending dosing program to IMD 12 from memory 86 via telemetry module 88. IMD 12 stores the pending dosing program to memory 40 of IMD 12. Programmer 20 may select a dosing program that is encoded in memory 40 of IMD 12 to activate the dosing program (360). The activated dosing program becomes the new current dosing program. That is, IMD 12 may begin administering fluid to patient 16 according to the activated dosing program, which is now the current dosing program (362).

In accordance with the techniques described in this disclosure, the clinician may quickly view and compare pending dosing programs against a current dosing program. Therefore, the clinician may decide whether a modification to the current dosing program, represented by a pending dosing program, presents a potential improvement in a therapy delivery schedule to be administered to a patient. User interface 82 of programmer 20 may provide an intuitive graphical representation of each of the dosing programs and/or therapy schedules of dosing programs. Programmer 20 may also facilitate quick, efficient evaluation of pending dosing programs vis-á-vis current dosing programs.

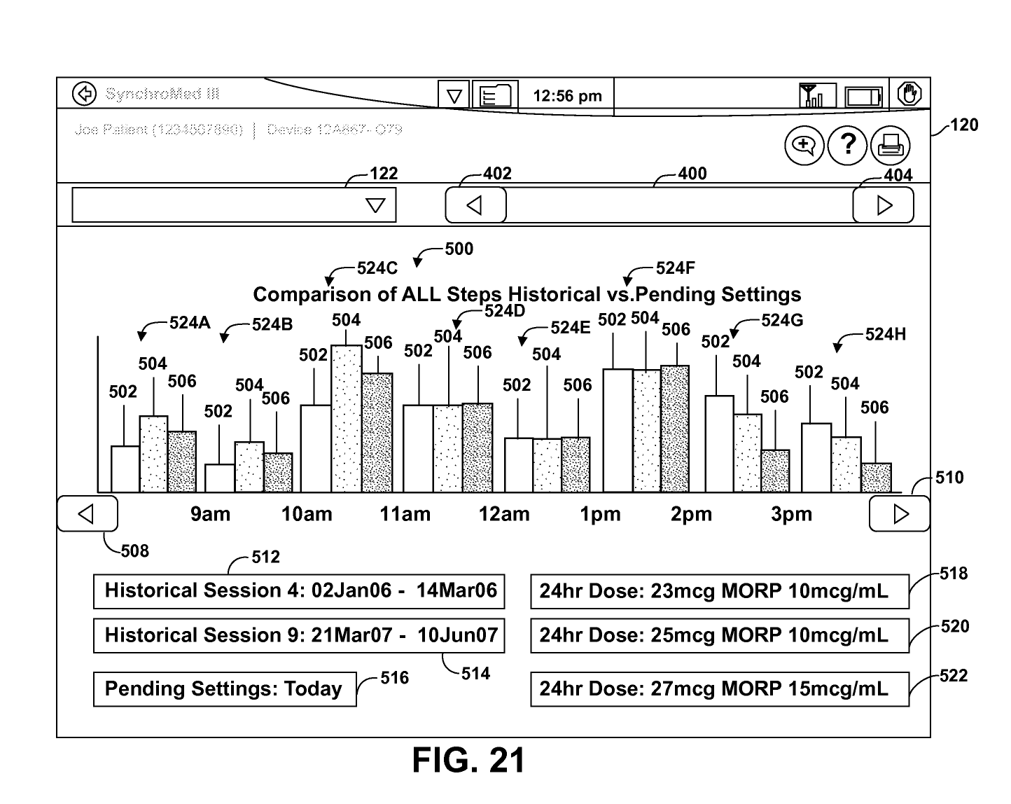

FIG. 21 is a screenshot illustrating an example user interface for displaying three dosing programs simultaneously. In the example of FIG. 21, processor 84 retrieves a first historical dosing program, a second historical dosing program, and a pending dosing program for IMD 12. Processor 84 causes user interface screen 120 to display graph 500, which includes representations of doses for each of the first historical dosing program, the second historical dosing program, and the pending dosing program. In particular, graph 500 includes first historical dosing program representations 502, second historical dosing program representations 504, and pending dosing program representations 506. Each of the first historical dosing program and the second historical dosing program may be considered a selected dosing program, in accordance with the techniques described herein.

In the example of FIG. 21, user interface screen 120 displays groupings 524A-524H (groupings 524) of representations of each of the dosing programs corresponding to an hour of the dosing programs. That is, for each hour of a therapy period, user interface screen 120 presents one of groupings 524 that include one of each of representations 502, 504, 506 corresponding to the respective dosing program of the representation. For example, grouping 524A includes representations 502, 504, 506 for the first historical dosing program, the second historical dosing program, and the pending dosing program, respectively. User interface screen 120 also presents left arrow 508 and right arrow 510, which allow the user to navigate to earlier hours or later hours, respectively.

User interface screen 120 also displays other information regarding the first historical dosing program, the second historical dosing program, and the pending dosing program. For example, user interface screen 120 represents that IMD 12 administered fluid according to the first historical dosing program from Jan. 2, 2006 to Mar. 14, 2006 with text field 512. User interface screen 120 also represents that IMD 12 administered fluid according to the second historical dosing program from Mar. 21, 2007 to Jun. 10, 2007 with text field 514. User interface screen 120 represents that IMD 12 will administer fluid according to the pending dosing program today with text field 516, e.g., assuming the user causes programmer 20 to program IMD 12 according to the pending dosing program. User interface screen 120 also represents that the total 24 hour dose was 23 mcg for the first historical dosing program, 23 mcg for the first historical dosing program, 25 mcg for the second historical dosing program, and will be 27 mcg for the pending dosing program with text fields 518, 520, and 522, respectively.

The techniques described in this disclosure may be implemented, at least in part, in hardware, software, firmware or any combination thereof. For example, various aspects of the described techniques may be implemented within one or more processors, which may be included in programmer 20, including one or more microprocessors, digital signal processors (DSPs), application specific integrated circuits (ASICs), field programmable gate arrays (FPGAs), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry.

Such hardware, software, and firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

The techniques described herein may also be embodied in a computer-readable medium, such as a computer-readable storage medium, containing or encoded with instructions. Instructions embedded or encoded in a computer-readable medium may cause a programmable processor, or other processor, to perform the method, e.g., when the instructions are executed. Computer readable storage media may include random access memory (RAM), read only memory (ROM), programmable read only memory (PROM), erasable programmable read only memory (EPROM), electronically erasable programmable read only memory (EEPROM), flash memory, a hard disk, a CD-ROM, a floppy disk, a cassette, magnetic media, optical media, or other computer readable media.

Various examples have been described. These and other examples are within the scope of the following claims.

1. A method comprising:
displaying, with a device for programming an implantable fluid delivery device, a first indication of a selected dosing program of the implantable fluid delivery device; and
displaying, simultaneously with the first indication, a second indication of a pending dosing program of the implantable fluid delivery device.

2. The method of claim 1,
wherein displaying a first indication comprises displaying the first indication as a first graphical representation of the selected dosing program, and
wherein displaying a second indication comprises displaying the second indication as a second graphical representation of the pending dosing program.

3. The method of claim 2,
wherein displaying a first graphical representation comprises displaying the first graphical representation with a first line style, and
wherein displaying a second graphical representation comprises displaying the second graphical representation with a second line style.

4. The method of claim 3,
wherein displaying the first graphical representation with a first line style comprises displaying the first graphical representation with a solid line, and
wherein displaying the second graphical representation with a second line style comprises displaying the second graphical representation with a dashed line.

5. The method of claim 2,
wherein displaying a first graphical representation comprises displaying the first graphical representation with a first color, and
wherein displaying a second graphical representation comprises displaying the second graphical representation with a second color.

6. The method of claim 2,
wherein displaying a first graphical representation comprises displaying the first graphical representation with a first shading style, and
wherein displaying a second graphical representation comprises displaying the second graphical representation with a second shading style.

7. The method of claim 2, further comprising:
displaying a first textual representation of the selected dosing program in conjunction with the first graphical representation; and
displaying a second textual representation of the pending dosing program in conjunction with the second graphical representation.

8. The method of claim 2,
wherein displaying a first graphical representation comprises displaying a first graph representative of the selected dosing program, wherein the first graph comprises a plurality of contiguous rectangles, each of the plurality of contiguous rectangles comprising a height representative of a rate of fluid delivery and a width representative of a time during which the rate is to be delivered.

9. The method of claim 2,
wherein displaying a second graphical representation comprises displaying a second graph representative of the pending dosing program, wherein the second graph comprises a plurality of contiguous rectangles, each of the plurality of contiguous rectangles comprising a height representative of a rate of fluid delivery and a width representative of a time during which the rate is to be delivered.

10. The method of claim 2, further comprising displaying, simultaneously with the first indication and the second indication, a third indication a third dosing program of the implantable fluid delivery device.

11. The method of claim 10, wherein the third dosing program comprises a historical dosing program.

12. The method of claim 10, wherein the second graphical representation comprises a graphical representation of a first therapy schedule of the pending dosing program, wherein displaying a third indication of a third dosing program comprises displaying the third indication of the third set of dosage information for an alternative therapy schedule of the pending dosing program, the method further comprising:
programming, with the programmer device, the implantable fluid delivery device to deliver fluid according to the first therapy schedule of the pending dosing program during a first time interval, and
programming the implantable fluid delivery device to deliver the fluid according to the alternative therapy schedule of the pending dosing program during a second time interval.

13. The method of claim 1, further comprising programming, with the programmer device, the implantable fluid delivery device to deliver fluid according to the pending dosing program.

14. A programmer device comprising:
a user interface comprising a display to present a graphical representation of doses of fluid to be delivered to a patient via an implantable fluid delivery device; and
a processor that controls the user interface to simultaneously present on the display a first indication of a selected dosing program of the implantable fluid delivery device and a second indication of a pending dosing program of the implantable fluid delivery pump.

15. The device of claim 14, wherein the user interface receives the pending dosing program from a user.

16. The device of claim 14, further comprising a telemetry module that communicates with the implantable fluid delivery device, wherein the telemetry module transmits the pending dosing program to the implantable fluid delivery device.

17. The device of claim 14, wherein the processor causes the user interface to present on the display the first indication as a first graphical representation of the selected dosing program and the second indication as a second graphical representation of the pending dosing program.

18. The device of claim 17, wherein the first graphical representation comprises a first line style, and wherein the second graphical representation comprises a second line style

19. The device of claim 17, wherein the first graphical representation comprises a first color, and wherein the second graphical representation comprises a second color.

20. The device of claim 17, wherein the first graphical representation comprises a first shading style, and wherein the second graphical representation comprises a second shading style.

21. The device of claim 17, wherein the user interface presents on the display a first textual representation of the selected dosing program in conjunction with the first graphical representation and a second textual representation of the pending dosing program in conjunction with the second graphical representation.

22. The device of claim 17,
wherein the first graphical representation comprises a first graph comprising a first plurality of contiguous rectangles, each of the first plurality of contiguous rectangles comprising a height representative of a rate of fluid delivery and a width representative of a time during which the rate is to be delivered, and
wherein the second graphical representation comprises a second graph comprising a second plurality of contiguous rectangles, each of the second plurality of contiguous rectangles comprising a height representative of a rate of fluid delivery and a width representative of a time during which the rate is to be delivered.

23. The device of claim 14, wherein the user interface displays a third indication of a third set of dosage information for a third dosing program of the implantable fluid delivery pump.

24. A system comprising:
an implantable fluid delivery pump that delivers fluid to a patient according to a selected dosing program, comprising a telemetry module; and
a programmer device comprising:
a user interface comprising a display to present a graphical representation of doses of fluid to be delivered to a patient via an implantable fluid delivery device; and
a processor that controls the user interface to simultaneously present on the display a first indication of a selected dosing program of the implantable fluid delivery device and a second indication of a pending dosing program of the implantable fluid delivery pump.

25. The system of claim 24, wherein the user interface of the programmer device receives the pending dosing program from a user.

26. The system of claim 24, wherein the programmer device further comprises a telemetry module, wherein the telemetry module of the programmer device transmits the pending dosing program to the telemetry module of the implantable fluid delivery device.

27. The system of claim 26, wherein the implantable fluid delivery device comprises a computer-readable medium that stores the selected dosing program, wherein the telemetry module of the implantable fluid delivery device receives the pending dosing program from the programmer device, and wherein the implantable fluid delivery device stores the pending dosing program in the computer-readable medium.

28. The system of claim 24, wherein the user interface of the programmer device displays the first indication as a first graphical representation of the selected dosing program and displays the second indication as a second graphical representation of the pending dosing program.

29. The system of claim 28, wherein the first graphical representation comprises a first line style, and wherein the second graphical representation comprises a second line style

30. The system of claim 28, wherein the first graphical representation comprises a first color, and wherein the second graphical representation comprises a second color.

31. The system of claim 28, wherein the first graphical representation comprises a first shading style, and wherein the second graphical representation comprises a second shading style.

32. The system of claim 28, wherein the user interface displays a first textual representation of the selected dosing program in conjunction with the first graphical representation and displays a second textual representation of the pending dosing program in conjunction with the second graphical representation.

33. The system of claim 28,
wherein the first graphical representation comprises a first graph comprising a first plurality of contiguous rectangles, each of the first plurality of contiguous rectangles comprising a height representative of a rate of fluid delivery and a width representative of a time during which the rate is to be delivered, and
wherein the second graphical representation comprises a second graph comprising a second plurality of contiguous rectangles, each of the second plurality of contiguous rectangles comprising a height representative of a rate of fluid delivery and a width representative of a time during which the rate is to be delivered.

34. The system of claim 24, wherein the user interface of the programmer device displays a third indication of a third set of dosage information for a third dosing program of the implantable fluid delivery pump.

35. A device comprising:
means for displaying, with a device for programming an implantable fluid delivery device, a first indication of a selected dosing program of the implantable fluid delivery device; and
means for displaying, simultaneously with the first indication, a second indication of a pending dosing program of the implantable fluid delivery device.

36. The device of claim 35, wherein the means for displaying the first indication comprise means for displaying the first indication as a first graphical representation of the selected dosing program, and wherein the means for displaying the second indication comprise means for displaying the second indication as a second graphical representation of the pending dosing program.

37. The device of claim 36,
wherein the first graphical representation comprises a first graph comprising a first plurality of contiguous rectangles, each of the first plurality of contiguous rectangles comprising a height representative of a rate of fluid delivery and a width representative of a time during which the rate is to be delivered, and
wherein the second graphical representation comprises a second graph comprising a second plurality of contiguous rectangles, each of the second plurality of contiguous rectangles comprising a height representative of a rate of fluid delivery and a width representative of a time during which the rate is to be delivered.

38. A computer-readable medium encoded with instructions for causing a programmable processor to:
display a first indication of a selected dosing program of an implantable fluid delivery pump; and
display, simultaneously with the first indication, a second indication of a pending dosing program of the implantable fluid delivery pump.

39. The computer-readable medium of claim 38, further comprising instructions to display the first indication as a first graphical representation of the selected dosing program and to display the second indication as a second graphical representation of the pending dosing program.

40. The computer-readable medium of claim 39,
wherein the instructions to display the first indication as a first graphical representation comprise instructions to display the first graphical representation as a first graph comprising a first plurality of contiguous rectangles, each of the first plurality of contiguous rectangles comprising a height representative of a rate of fluid delivery and a width representative of a time during which the rate is to be delivered, and
wherein the instructions to display the second indication as a second graphical representation comprise instructions to display the second graphical representation as a second graph comprising a second plurality of contiguous rectangles, each of the second plurality of contiguous rectangles comprising a height representative of a rate of fluid delivery and a width representative of a time during which the rate is to be delivered.
